(19) United States
(12) Patent Application Publication
(10) Pub. No.: US 2026/0199534 A1
(43) Pub. Date: Jul. 16, 2026

(54) COMPOUNDS FOR RADIOIMAGING AND TREATMENT OF A CANCER

(71) Applicant: Clarity Pharmaceuticals Limited, Eveleigh, New South Wales (AU)

(72) Inventors: Matthew John HARRIS, Eveleigh, New South Wales (AU); Ellen Marianne VAN DAM, Eveleigh, New South Wales (AU); Lachlan Eion MCINNES, Eveleigh, New South Wales (AU)

(21) Appl. No.: 19/127,882

(22) PCT Filed: Nov. 10, 2023
(86) PCT No.: PCT/AU2023/051143

§ 371 Date: May 7, 2025

(30) Foreign Application Priority Data
Nov. 11, 2022 (AU) 2022903382

(51) Int. Cl. A61K 51/04; C07B 59/00

(52) U.S. Cl. CPC A61K 51/0497; A61K 51/0455; C07B 59/002; A61K 2121/00; A61K 2123/00; C07B 2200/05

(57) ABSTRACT

The present invention relates to compounds that inhibit fibroblast activation protein (FAP) and their uses in the treatment, prevention and/or imaging of a cancer associated with the expression of fibroblast activation protein.

### FIELD

The present invention relates to compounds that inhibit fibroblast activation protein (FAP) and their uses in the treatment, prevention and/or imaging of a cancer associated with the expression of fibroblast activation protein.

### BACKGROUND

Fibroblast activation protein (FAP) is a type II transmembrane serine protease with dipeptidyl peptidase and endopeptidase activity. FAP is also known as FAP-alpha, seprase or alpha2 antiplasmin converting enzyme and is typically expressed at low levels in healthy human tissue. In contrast, FAP is often overexpressed in fibroblasts of various cancers and proliferative diseases. It is thought that FAP plays a role in various tumour-promoting activities, such as matrix remodelling, angiogenesis, chemotherapy resistance and immunosuppression.

FAP is known to have enzyme activity as a peptidase, which contributes to matrix digestion and remodelling of the tumour microenvironment. This then facilitates the invasion and migration of tumour cells. Neuropeptide Y is a known substrate of FAP and since the cleavage product of neuropeptide Y in the presence of FAP is proangiogenic, FAP is considered to contribute to tumour angiogenesis. In addition to its enzyme activity, FAP also has a role in cell signalling by forming complexes with other proteins.

Fibroblasts expressed by cancerous cells are distinct from fibroblasts associated with healthy cells. Since there is a marked difference in the expression of FAP between healthy and cancerous tissue, treatment of cancers by administering compounds that show selectivity for FAP may be an alternative to current cancer treatments.

However in order for a treatment regime to be useful for the treatment of cancer and related disorders, the agent administered must first show sufficient selectivity for FAP over other receptor sites present in other tissues. Even if a compound is shown to be selective for FAP, the compound must be retained at the relevant site for a sufficient time. Compounds known to show selectivity for FAP often show limited retention at the cancer site and are therefore unsuitable for use as part of a treatment regime for the associated cancer.

Furthermore, where the compound selective for FAP is used for radiotherapy and/or radioimaging, the compound must be able to coordinate, retain and transport the selected radioisotope to the desired site without significant decomposition of the compound or loss of the radioisotope.

There remains a need for compounds that are suitable for use in radiotherapy and/or radioimaging, where the compounds are selective for FAP associated with a cancer or related disorder, have sufficient retention at the cancer site and have the requisite chemical stability and pharmacokinetic profile.

### SUMMARY OF THE INVENTION

The present inventors have found that the compounds disclosed herein are able to bind FAP with inhibition. The compounds disclosed herein comprise a fragment that is capable of binding to FAP and a sarcophagine, which is capable of coordinating and retaining a metal ion (for example, a radioisotope), where the FAP binding inhibitor and the sarcophagine are joined by a linker. Since the compounds of the present invention can both bind to FAP and deliver a radioisotope, the inventors believe that the compounds of the present invention may be useful in the selective targeting of malignant sites that overexpress FAP and subsequently deliver a dose of radioactivity provided by the accompanying radioisotope to the cancer site.

In a first aspect, the present invention provides a compound of Formula (I), or a salt, complex, isomer, solvate or prodrug thereof:

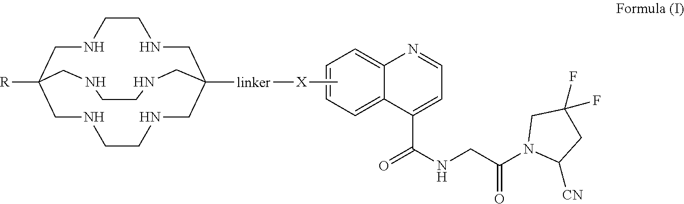

wherein:
R is a group selected from the group consisting of H, OH, halogen, cyano, NO₂, NH₂, optionally substituted C₁-C₁₂ alkyl, optionally substituted amino, optionally substituted amide, optionally substituted aryl and a group having the following structure:

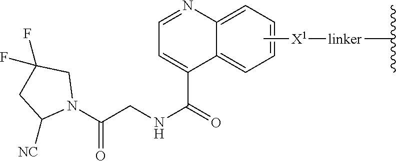

wherein:
X and X¹ may be the same or different and are independently selected from the group consisting of O, S, NH and N—(C₁₋₁₂alkyl).

In certain embodiments, the linker in the compound of Formula (I) comprises one or more moieties selected from the group consisting of:
optionally substituted C₁-C₁₂ alkylene, wherein one or more alkylene groups may be replaced with O, S, NH or N—(C₁₋₁₂alkyl);

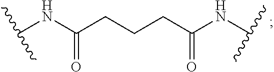 [SMILES: *NC(=O)CCCC(=O)N*]

one or more amino acids;

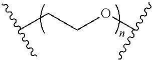

wherein n is an integer from 1 to 10; and

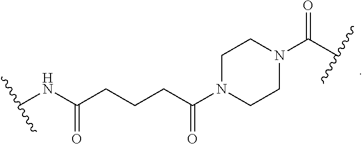 [SMILES: *NC(=O)CCCC(=O)N1CCN(C(*)=O)CC1]

In some embodiments, the linker in the compound of Formula (I) comprises the group:

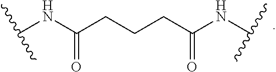 [SMILES: *NC(=O)CCCC(=O)N*]

In some embodiments, the linker in the compound of Formula (I) is selected from the group consisting of:

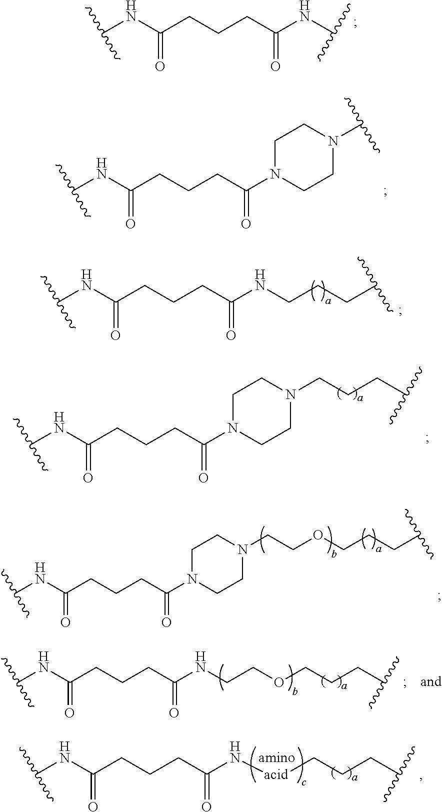

wherein a, b and c are integers independently selected from 1 to 10.

In certain embodiments, R is H, NH₂, optionally substituted C₁-C₁₂ alkyl or optionally substituted amide. In specific embodiments, R is an optionally substituted amide having the following structure:

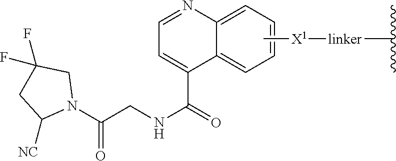

wherein:
X¹ is O, S, NH or N—(C₁₋₁₂alkyl); and
the linker comprises one or more moieties selected from the group consisting of:

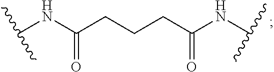 [SMILES: *NC(=O)CCCC(=O)N*]

optionally substituted C₁-C₁₂ alkylene, wherein one or more alkylene groups may be replaced with O, S, NH or N—(C₁₋₁₂alkyl);
one or more amino acids;

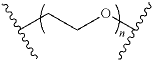

wherein n is an integer from 1 to 10; and

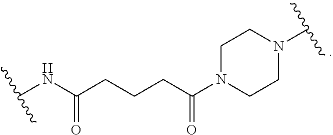 [SMILES: *NC(=O)CCCC(=O)N1CCN(*)CC1]

In certain embodiments, the compound of Formula (I) has the structure of Formula (Ia);

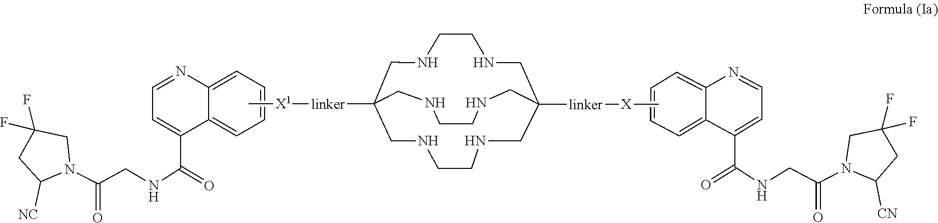

wherein
X and X¹ may be the same or different and are independently selected from the group consisting of O, S, NH and N—(C₁₋₁₂alkyl); and
the linker comprises one or more moieties selected from the group consisting of:

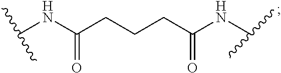 [SMILES: *NC(=O)CCCC(=O)N*]

optionally substituted C₁-C₁₂ alkylene, wherein one or more alkylene groups may be replaced with O, S, NH or N—(C₁₋₁₂alkyl);
one or more amino acids;

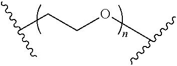

wherein n is an integer from 1 to 10; and

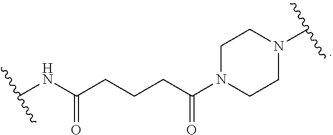 [SMILES: *NC(=O)CCCC(=O)N1CCN(*)CC1]

In some embodiments, the linker in the compound of Formula (Ia) comprises the group:

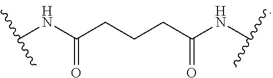 [SMILES: *NC(=O)CCCC(=O)N*]

In some embodiments, the linkers in the compound of Formula (Ia) may be the same or different and are independently selected from the group consisting of:

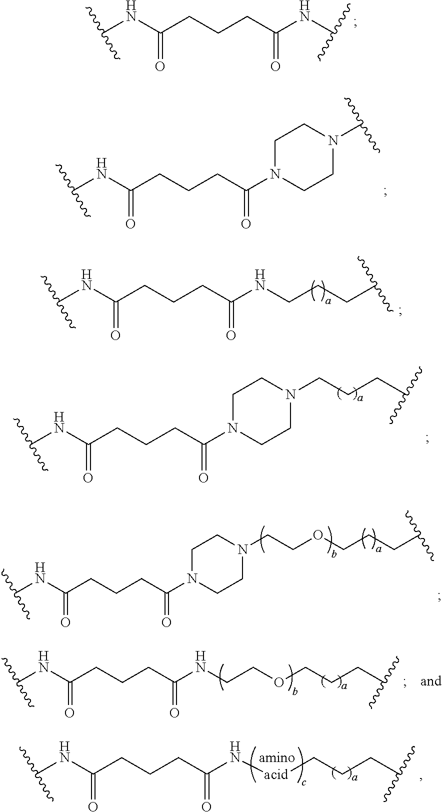

wherein a, b and c are integers independently selected from 1 to 10.

In certain embodiments, the compound of Formula (I) is Sar-FAPi and has the following structure:

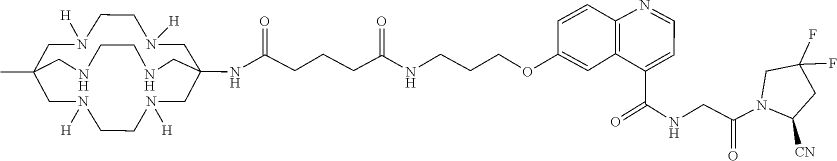 [SMILES: CC12CNCCNCC(NC(=O)CCCC(=O)NCCCOc3ccc4nccc(C(=O)NCC(=O)N5CC(F)(F)C[C@H]5C#N)c4c3)(CNCCNC1)CNCCNC2]

In certain embodiments, the compound of Formula (Ia) is Sar-bisFAPi and has the following structure:

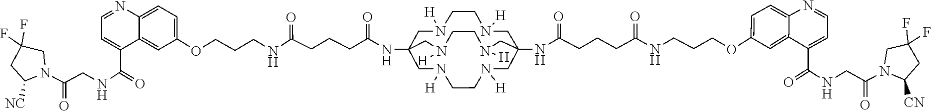 [SMILES: N#C[C@@H]1CC(F)(F)CN1C(=O)CNC(=O)c1ccnc2ccc(OCCCNC(=O)CCCC(=O)NC34CNCCNCC(NC(=O)CCCC(=O)NCCCOc5ccc6nccc(C(=O)NCC(=O)N7CC(F)(F)C[C@H]7C#N)c6c5)(CNCCNC3)CNCCNC4)cc12]

In certain embodiments, the compound of Formula (I) is complexed with a metal ion.

In some embodiments, the compound of Formula (I) is complexed with a radioisotope. In some embodiments, the radioisotope is a Cu radioisotope. In some embodiments, the radioisotope is selected from the group consisting of ⁶⁰Cu, ⁶¹Cu, ⁶²Cu, ⁶⁴Cu and ⁶⁷Cu.

The present inventors have now shown in FIGS. 9 and 10 that compounds of Formula (I) and (Ia) may be radiolabelled with a copper radioisotope. As shown in FIGS. 11 and 12, the radiolabelled compounds show exceptional stability and radiochemical purity at ambient temperature.

Radiolabelled compounds of Formula (I) and (Ia), specifically [⁶⁴Cu]Sar-FAPi and [⁶⁴Cu]Sar-bisFAPi were exposed to SK-MEL 187 (FAP-positive melanoma xenograft) and LNCaP cells. As seen in FIG. 13, the present inventors have found that the compounds of the present invention as disclosed herein show excellent binding affinity to cells expressing the FAP receptor.

In a second aspect, the present invention provides a composition comprising a compound according to the first aspect and a pharmaceutically acceptable excipient.

In a third aspect, the present invention provides a method for the treatment of a cancer, the method comprising administering to the subject in need thereof a compound of Formula (I) as defined in the first aspect, or a salt, complex, isomer, solvate or prodrug thereof, wherein the compound of Formula (I) contains a suitable radioisotope.

In certain embodiments, the cancer is associated with a fibroblast activation protein (FAP) receptor.

In a fourth aspect, the present invention provides a method for radioimaging a cancer, the method comprising administering to a subject in need thereof a compound of Formula (I) as defined in the first aspect, or a salt, complex, isomer, solvate or prodrug thereof, wherein the compound of Formula (I) contains a suitable radioisotope.

In certain embodiments of the third and fourth aspects, the radioisotope is a Cu radioisotope. In other embodiments, the Cu radioisotope is selected from the group consisting of ⁶⁰Cu, ⁶¹Cu, ⁶²Cu, ⁶⁴Cu and ⁶⁷Cu.

In certain embodiments, the cancer is selected from the group consisting of epithelial ovarian cancer, ovarian carcinoma, osteosarcoma, pancreatic adenocarcinoma, colorectal cancer, lung cancer, non-small cell lung cancer, gastric cancer, endometrial carcinoma, pancreatic adenocarcinoma, medullary thyroid carcinoma, differentiated thyroid cancer, breast cancer, invasive ductal carcinoma of the breast, oral squamous cell carcinoma, esophageal cancer, renal cell cancer, insulinoma, prostate cancer, neuroendocrine differentiated prostate cancer, pheochromocytoma, adenoid cystic cancer, hepatocellular carcinoma, cervical cancer, small intestine cancer, neuroendocrine tumour, anal cancer, chordoma, desmoid tumour, head and neck cancer, thymus cancer, pancreatic cancer, cholangiocellular carcinoma, esophageal cancer, salivary gland cancer, sarcoma and carcinoma of unknown primary cancer.

In a fifth aspect, the present invention provides the use of a compound of Formula (I), or a salt, complex, isomer, solvate or prodrug thereof, in the manufacture of a medicament for the treatment of a cancer.

In a sixth aspect, the present invention provides a kit comprising:
i) a container comprising a compound of Formula (I) as defined in the first aspect, or a pharmaceutically acceptable salt thereof;
ii) a container comprising a solution of a Cu ion; and
iii) instructions for preparing an aqueous formulation of a compound of Formula (I), or a pharmaceutically acceptable salt thereof, complexed with a Cu ion.

In certain embodiments, the Cu ion provided as part of a kit is a Cu radioisotope. In certain embodiments, the radioisotope is selected from the group consisting of ⁶⁰Cu, ⁶¹Cu, ⁶²Cu, ⁶⁴Cu and ⁶⁷Cu.

In a seventh aspect, the present invention provides a method for producing a compound of Formula (I) or a pharmaceutically acceptable salt thereof, the method comprising the step of:
i) preparing a compound of Formula A or a protected form thereof:

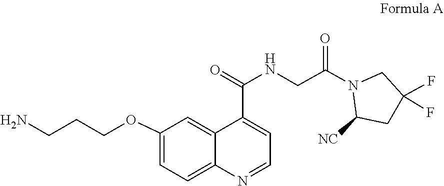 [SMILES: N#C[C@@H]1CC(F)(F)CN1C(=O)CNC(=O)c1ccnc2ccc(OCCCN)cc12]

ii) preparing a compound of Formula B or a protected form thereof:

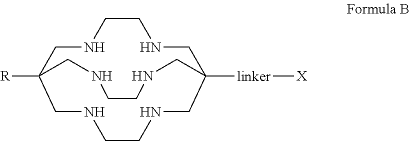

iii) coupling the compound of Formula A or a protected form thereof with the compound of Formula B or a protected form thereof,
   to produce the compound of Formula (I) or a protected form thereof,
   wherein:
R is a group selected from the group consisting of H, OH, halogen, cyano, NO₂, NH₂, optionally substituted C₁-C₁₂ alkyl, optionally substituted amino, optionally substituted amide, optionally substituted aryl and a group having the following structure:

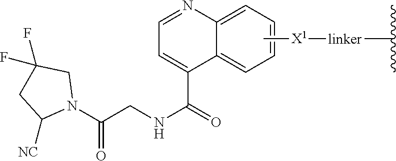

wherein:
X and X¹ may be the same or different and are independently selected from the group consisting of O, S, NH and N—(C₁₋₁₂alkyl).

In certain embodiments, the coupling of the compound of Formula A with a compound of Formula B occurs in the presence of a base.

### BRIEF DESCRIPTION OF THE FIGURES

FIG. 1. HPLC traces of Sar-FAPi with detection by UV-vis spectroscopy, absorbance at A) 254 nm, and B) 220 nm, showing purity of the synthesised compound.

FIG. 2. Fragmentation pattern of Sar-FAPi analysed by mass spectrometry. The signals detected correspond with the expected molecular mass (and fragments) of Sar-FAPi.

FIG. 3. HPLC trace of Sar-FAPi and [natCu]CuSar-FAPi with UV-vis detection at 280 nm. The different retention times indicate that coordination of Sar-FAPi with a Cu ion has occurred.

FIG. 4. RadioTLC of a solution of [64Cu]CuCl2 buffered with ammonium acetate. The presence of a single signal by TLC provides a standard retention for [64Cu]CuCl2, which then allows for comparisons to be made when the radioisotope is used to radiolabel Sar-FAPi.

FIG. 5. Analysis of Sar-FAPi (1 g) radiolabelled with 64Cu (4 MBq) by A) RadioHPLC and B: radioTLC. Analysis of the radiolabelling reaction by radioHPLC shows the appearance of a new signal when compared to radioHPLC analysis of the corresponding complex labelled with [natCu]. Analysis by radioTLC also shows the presence of the 64Cu radiolabelled Sar-FAPi compound.

FIG. 6. RadioHPLC trace of Sar-FAPi (10 ng) radiolabelled with 64Cu (4 MBq). Analysis of the radiolabelling reaction by radioHPLC shows the appearance of a new signal when compared to analysis of the corresponding complex labelled with [natCu].

FIG. 7. RadioHPLC of a reaction mixture containing Sar-FAPi (1 g) and [64Cu]CuCl2 (4 MBq) after 10 minutes and 23 hours. Comparison of the radioHPLC traces for the reaction mixtures after 10 minutes and 23 hours is substantially the same and does not contain any new signals corresponding to other 64Cu-containing species. This shows that Sar-FAPi radiolabelled with 64Cu maintains the same radiochemical purity for at least 23 hours and is stable for at least the same amount of time.

FIG. 8. HPLC traces of [64Cu]Cu-SarFAPi with the addition of either histidine or cysteine. Solutions of [⁶⁴Cu]Cu-SarFAPi were treated with either histidine hydrochloride or cysteine hydrochloride and analysed by HPLC. There was no change in retention time in either case, which indicates that the same species (i.e. [64Cu]Cu-SarFAPi) is present and no new species were formed (e.g. copper bound with either histidine or cysteine).

FIG. 9. RadioTLC traces of [⁶⁴Cu]2 buffered with A) PBS or B) NH4OAc showing retention of [⁶⁴Cu]CuCl₂; radioTLC traces of Sar-FAPi radiolabelled with [⁶⁴Cu]CuCl2 in C) PBS and E) NH4OAc, with the further addition of Sar-FAPi to each (see D) and F)). With the use of an EDTA (10 mM) and PBS as a mobile phase, retention of [⁶⁴Cu]CuCl2 was similar in both radiolabelling buffer systems (PBS and NH4OAc, see A) and B)), while introduction of [⁶⁴Cu]CuCl2 to a solution of Sar-FAPi (in either PBS or NH4OAc) showed the appearance of a new species, i.e. [⁶⁴Cu]Sar-FAPi. In both buffer systems, since unchelated copper was still present, a further aliquot of Sar-FAPi was added and analysed further by radioTLC ((see C) and E)). In both buffer systems, the signal representing ⁶⁴Cu-SarFAPi intensified, with complete chelation of the ⁶⁴Cu ion observed in the presence of NH₄OAc (i.e. disappearance of free ⁶⁴Cu.

FIG. 10. RadioTLC traces of [⁶⁴Cu]CuCl₂ buffered with A) PBS or B) NH4OAc showing retention of [⁶⁴Cu]CuCl₂; radioTLC traces of Sar-bisFAPi radiolabelled with [⁶⁴Cu]CuCl2 in C) PBS and E) NH4OAc, with the further addition of Sar-bisFAPi to each (see D) and F)). With the use of an EDTA (10 mM) and PBS as a mobile phase, retention of [⁶⁴Cu]CuCl₂ was similar in both radiolabelling buffer systems (PBS and NH4OAc, see A) and B)), while introduction of [⁶⁴Cu]CuCl₂ to a solution of Sar-bisFAPi (in either PBS or NH4OAc) showed the appearance of a new species, i.e. [⁶⁴Cu]Sar-bisFAPi. In both buffer systems, since unchelated copper was still present, a further aliquot of Sar-bisFAPi was added and analysed further by radioTLC ((see C) and E)). In both buffer systems, the signal representing ⁶⁴Cu-Sar-bisFAPi intensified, with complete chelation of the ⁶⁴Cu ion observed in the presence of NH₄OAc (i.e. disappearance of free ⁶⁴Cu).

FIG. 11. RadioTLC traces of [⁶⁴Cu]Sar-FAPi at A) 1 hour and B) 24 hours and radioHPLC traces of [⁶⁴Cu]Sar-FAPi at C) 1 hour and D) 24 hours. Solutions of [⁶⁴Cu]Sar-FAPi were analysed by radioTLC and radioHPLC at 1 hour and 24 hours after radiolabelling. The compound showed excellent stability with only about 5% of the ⁶⁴Cu radioisotope unchelated after 24 hours at ambient temperature.

FIG. 12. RadioTLC traces of [⁶⁴Cu]Sar-bisFAPi at A) 1 hour and B) 24 hours and radioHPLC traces of [⁶⁴Cu]Sar-bisFAPi at C) 1 hour and D) 24 hours. Solutions of [⁶⁴Cu]Sar-bisFAPi were analysed by radioTLC and radioHPLC at 1 hour and 24 hours after radiolabelling. The compound showed excellent stability with little to no free ⁶⁴Cu radioisotope detected after 24 hours at ambient temperature.

FIG. 13. Chart showing percentage of cells having bound [⁶⁴Cu]Sar-FAPi or [⁶⁴Cu]Sar-bisFAPi after incubation for 1 hour. After 1 hour, approximately 91% of cells showed binding with [⁶⁴Cu]Sar-bisFAPi, while at least 77% of cells showed binding with [⁶⁴Cu]Sar-FAPi.

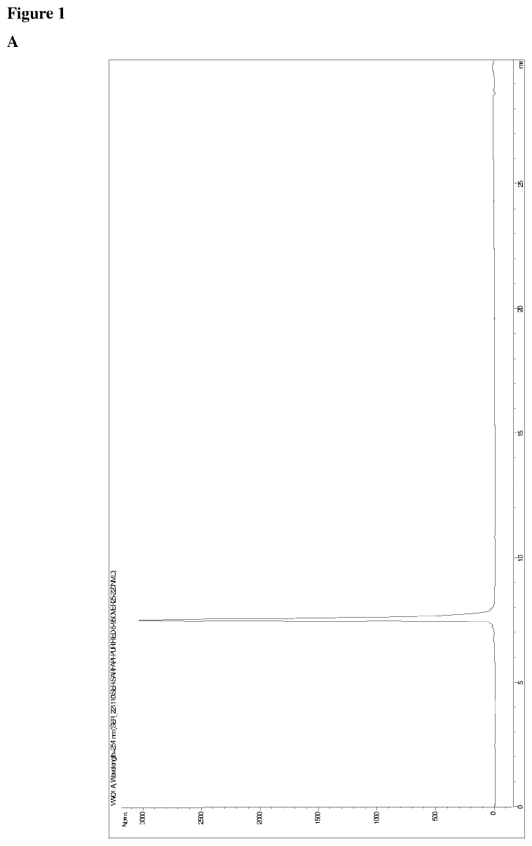

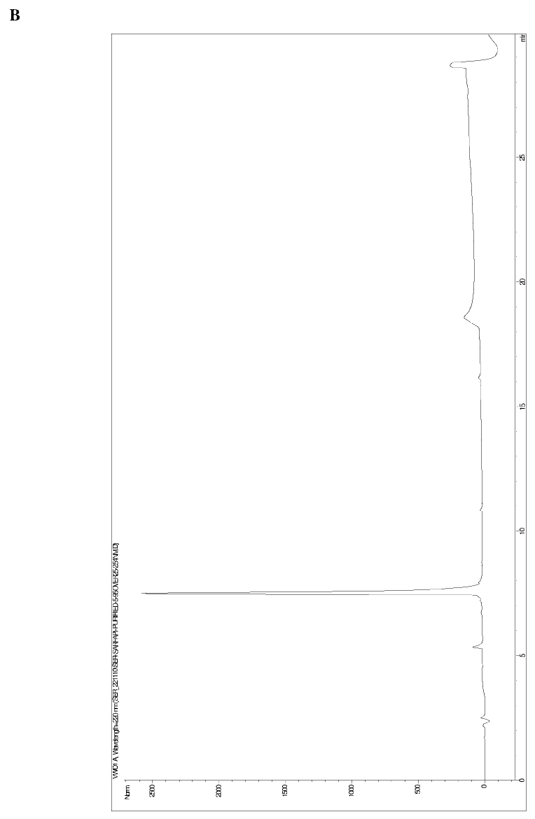

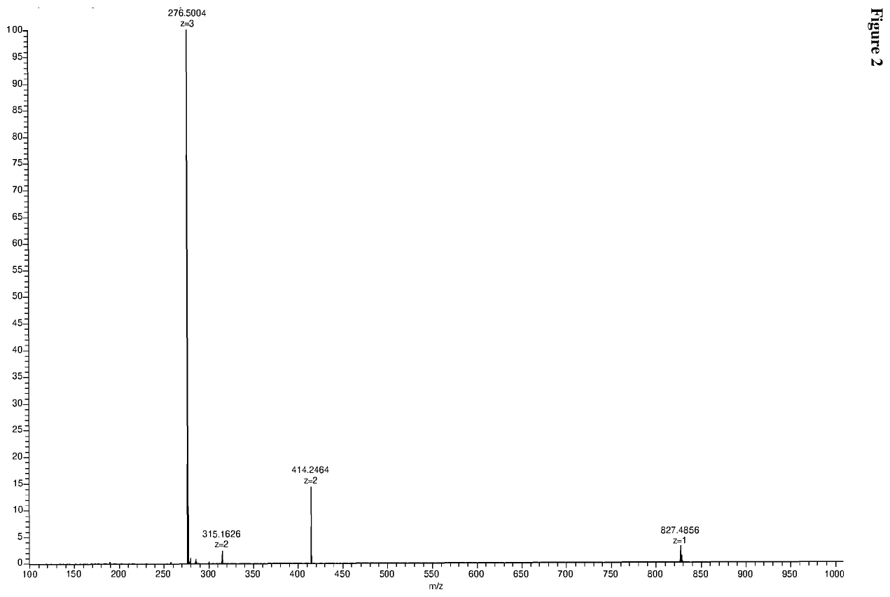

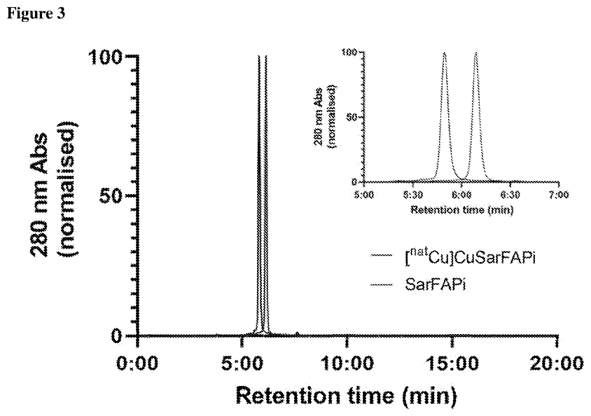

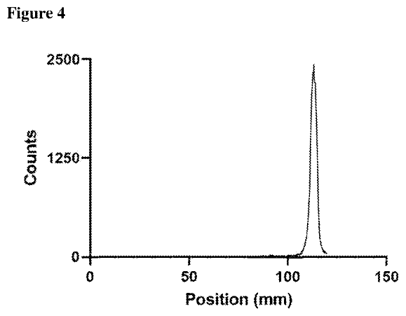

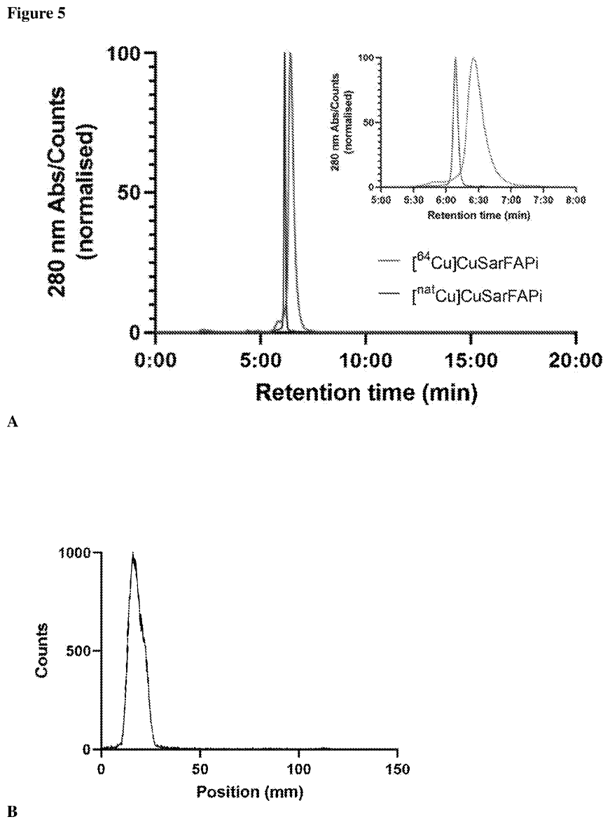

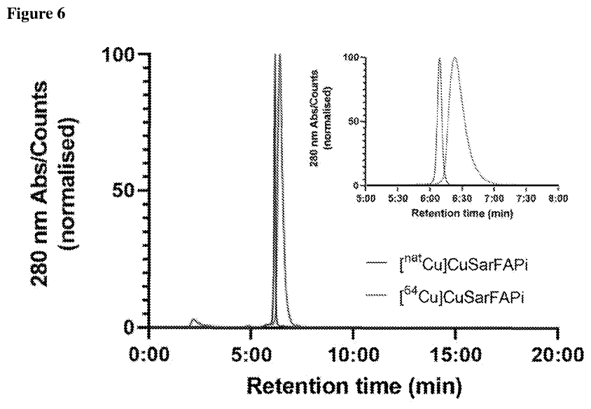

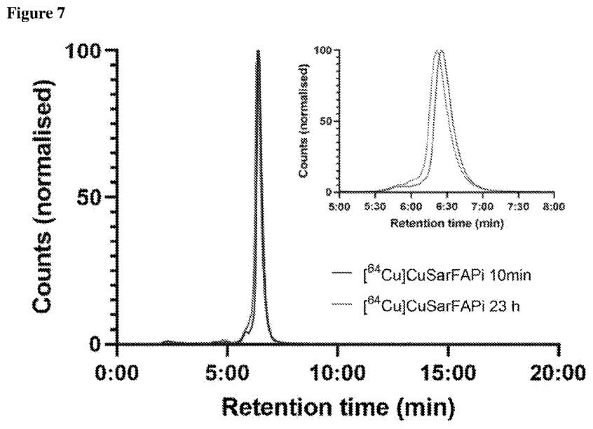

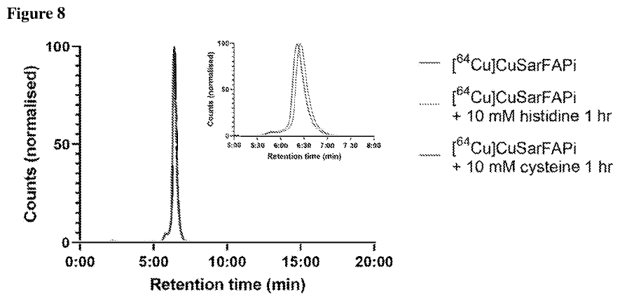

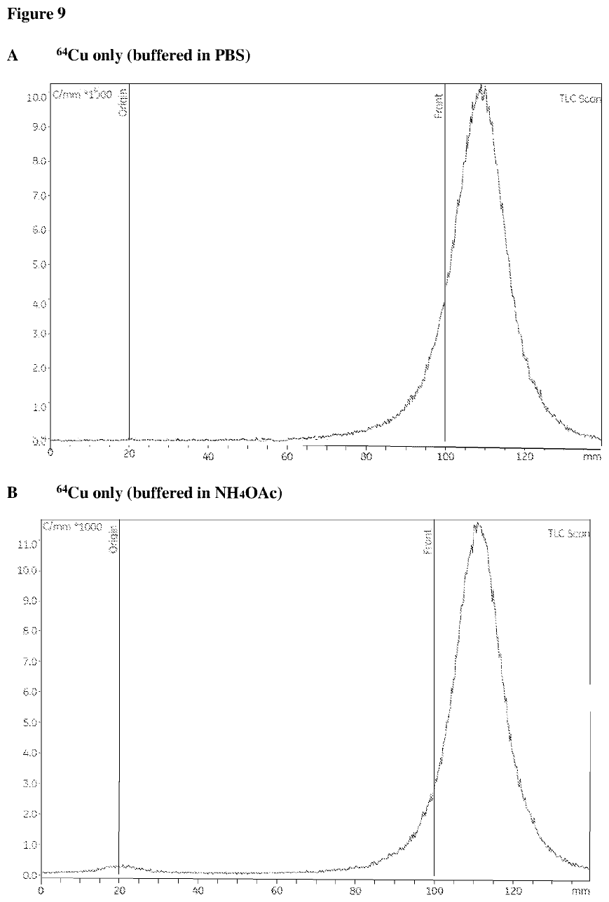

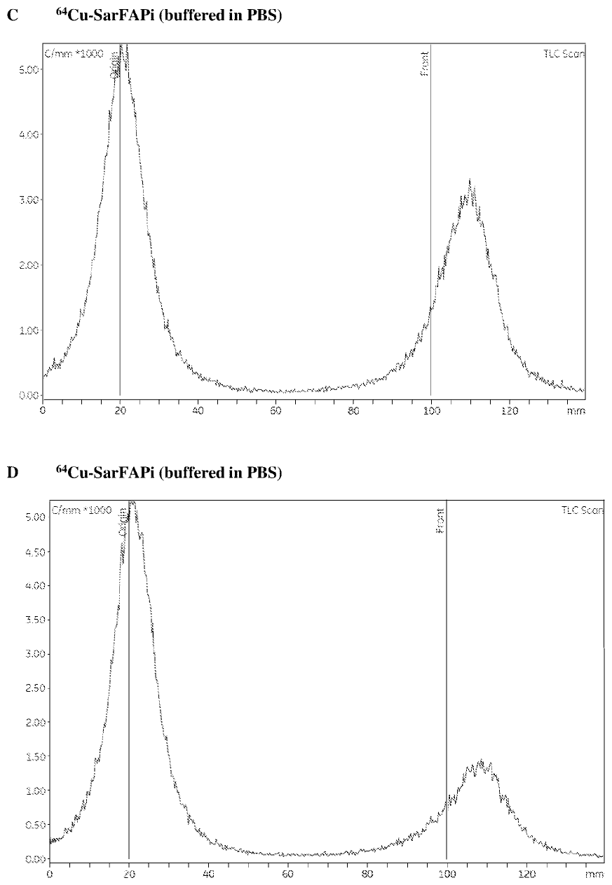

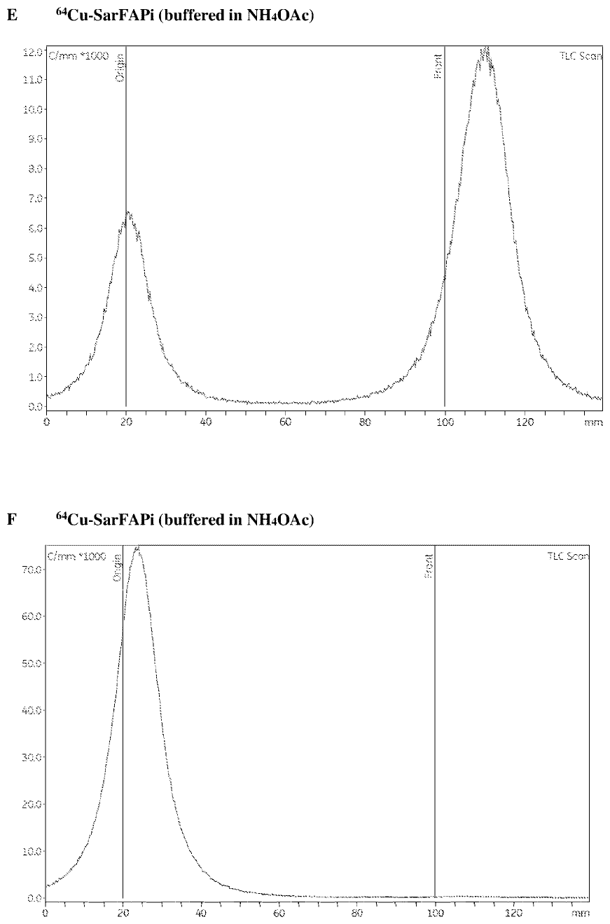

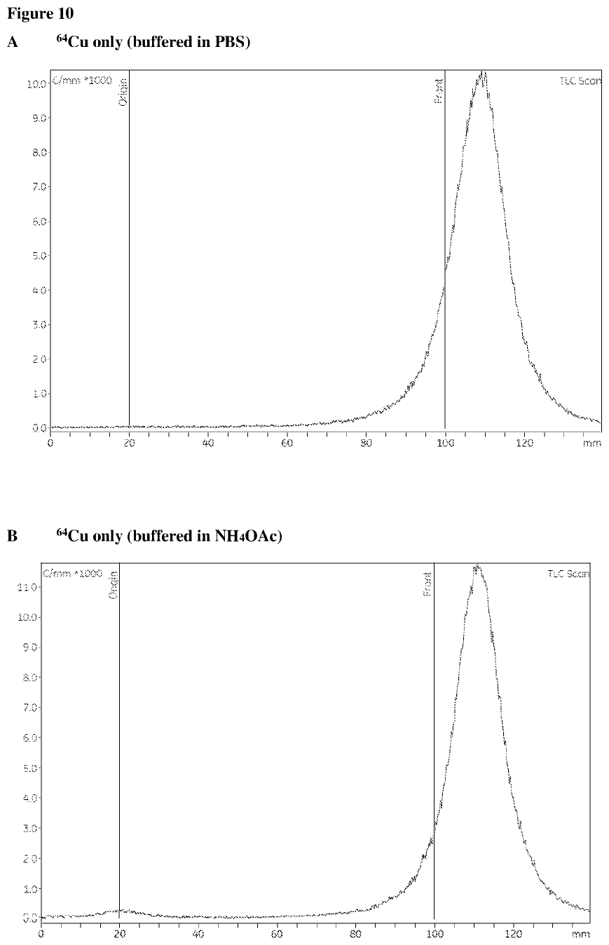

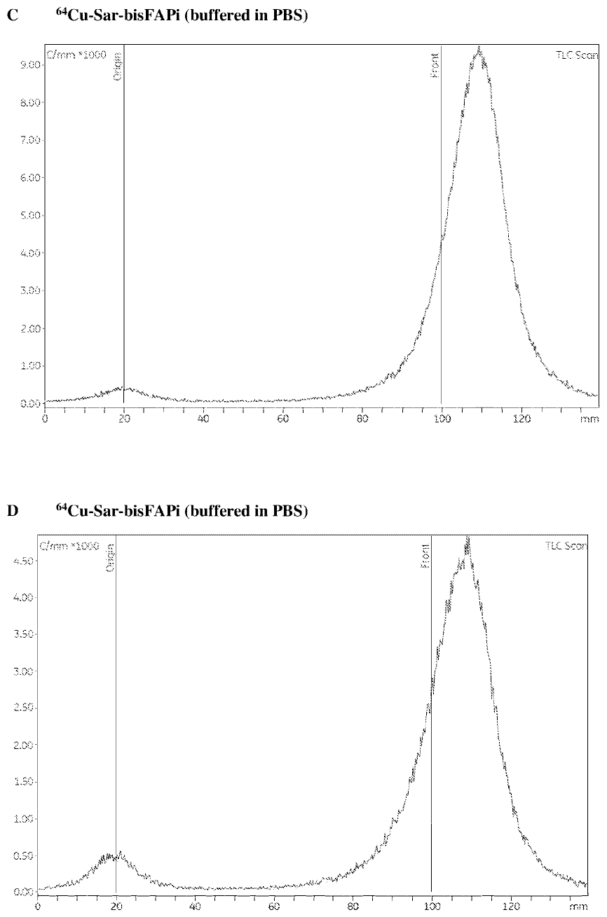

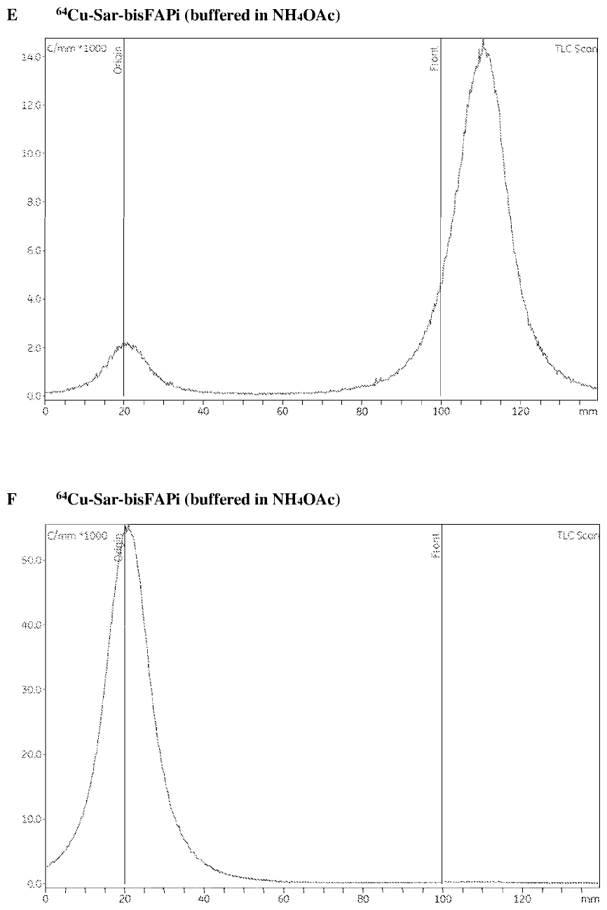

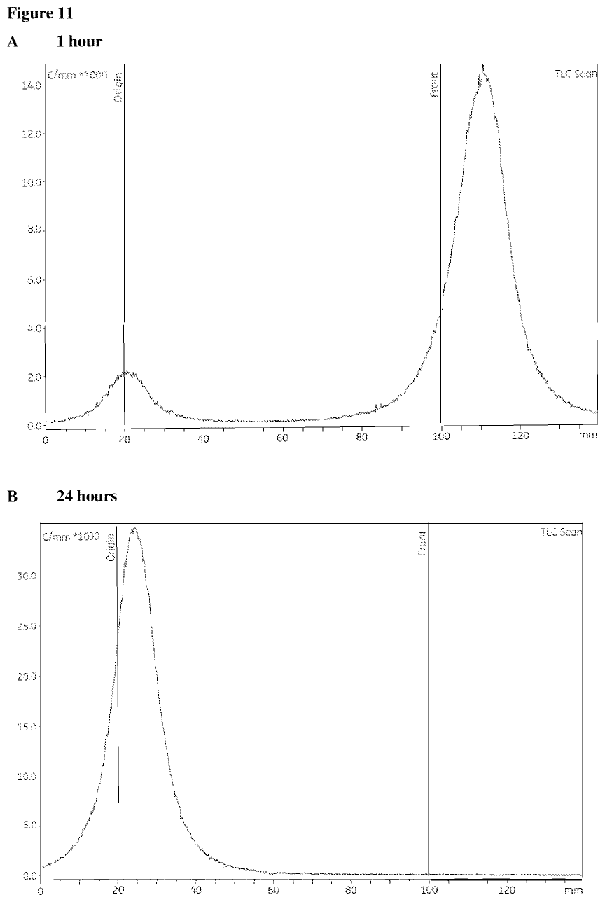

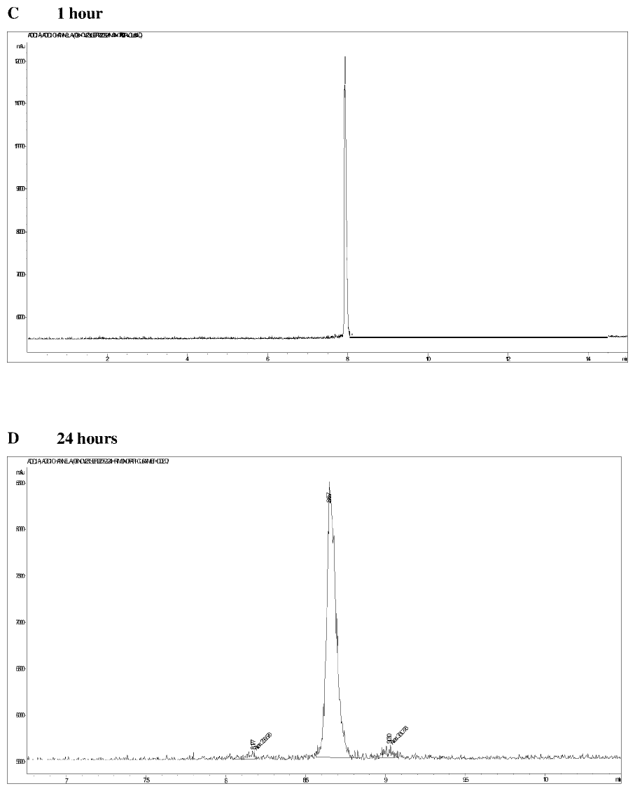

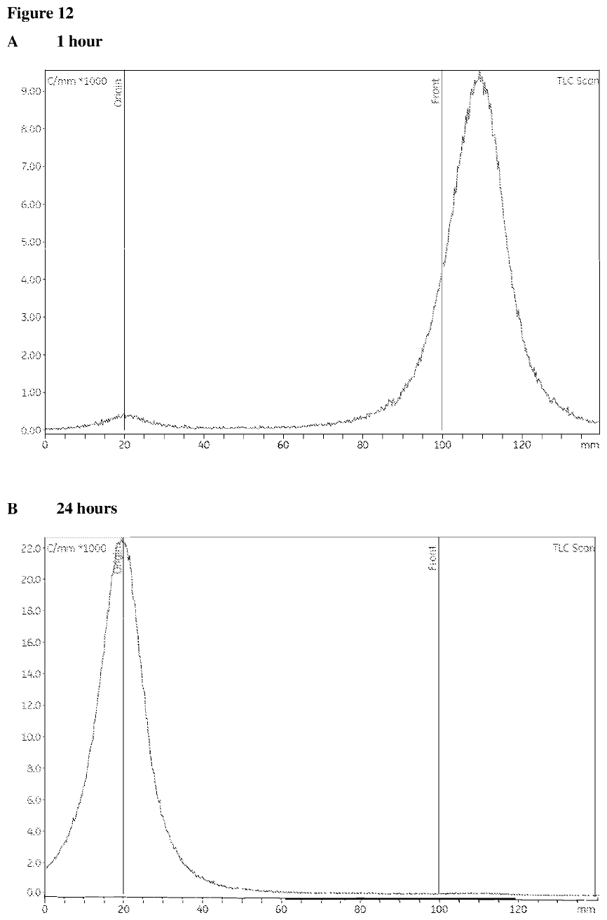

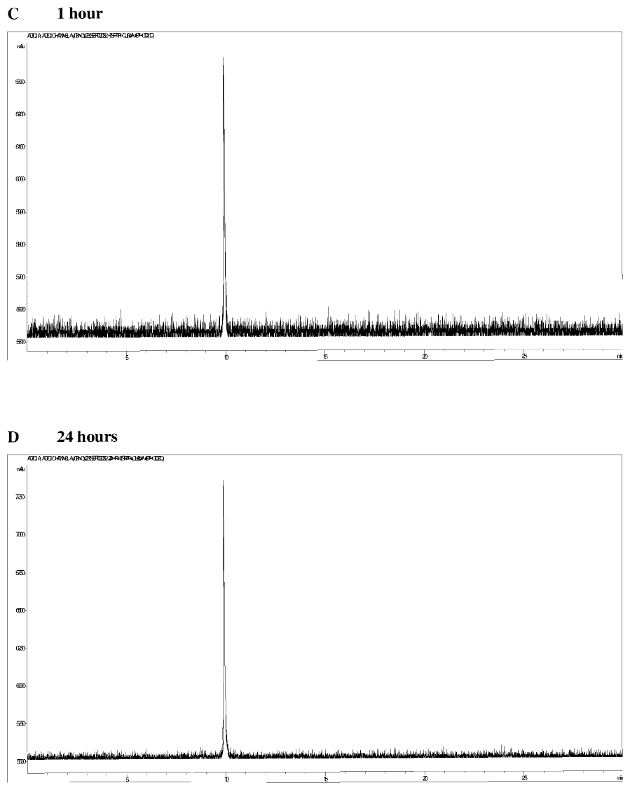

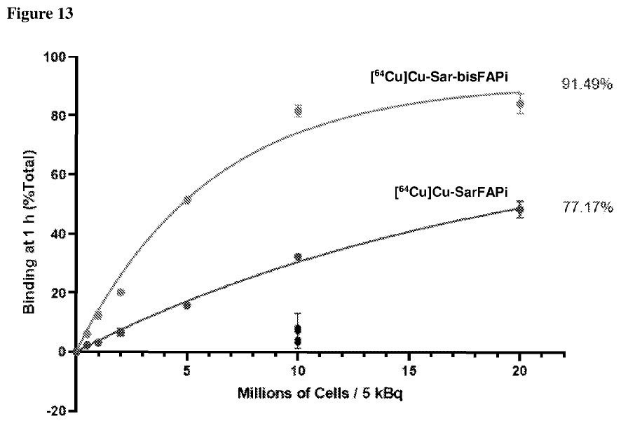

### DETAILED DESCRIPTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “about” or “approximately” as used herein means within an acceptable error range for the particular value as determined by one of ordinary skill in the art, which will depend in part on how the value is measured or determined, i.e., the limitations of the measurement system.

Unless defined otherwise, all technical and scientific terms used herein have the same meaning as commonly understood by those of ordinary skill in the art to which the invention belongs. For the purposes of the present invention, the following terms are defined below.

The term “pharmaceutically acceptable salts” refers to salts that retain the desired biological activity of the above-identified compounds, and include pharmaceutically acceptable acid addition salts and base addition salts. Suitable pharmaceutically acceptable acid addition salts of compounds of Formula (I) may be prepared from an inorganic acid or from an organic acid. Examples of such inorganic acids are hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic and arylsulfonic acids. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts may be prepared by reaction of a compound with the appropriate inorganic or organic acid via any of a number of known methods. Alternatively, alkali and alkaline earth metal salts can be prepared by reacting a compound with the appropriate base via a variety of known methods. The following are further examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates. Additional information on pharmaceutically acceptable salts can be found in Remington's Pharmaceutical Sciences, 19th Edition, Mack Publishing Co., Easton, PA 1995. In the case of agents that are solids, it is understood by those skilled in the art that the inventive compounds, agents and salts may exist in different crystalline or polymorphic forms, all of which are intended to be within the scope of the present invention and specified formulae.

As used herein, the term “sarcophagine” refers to the nitrogen-containing macrocyclic ligand with the formula 3,6,10,13,16,19-hexaazabicyclo[6.6.6]icosane.

As used herein, the term “optionally substituted” as used throughout the specification denotes that the group may or may not be further substituted or fused (so as to form a condensed polycyclic system), with one or more non-hydrogen substituent groups. In certain embodiments the substituent groups are one or more groups independently selected from the group consisting of halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalkylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkyloxy, alkyloxyalkyl, alkyloxycycloalkyl, alkyloxyheterocycloalkyl, alkyloxyaryl, alkyloxyheteroaryl, alkyloxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, —C(═O)OH, —C(═O)R^{a}, —C(═O)OR^{a}, C(═O)NR^{a}R, C(═NOH)R^{a}, C(═NR^{a})NR^{b}R^{c}, NR^{a}R^{b}, NR^{a}C(═O)R^{b}, NR^{a}C(═O)OR^{b}, NR^{a}C(═O)NR^{b}R^{c}, NR^{a}C(═NR^{b})NR^{c}R^{d}, NR^{a}SO₂R^{b}, —SR^{a}, SO₂NR^{a}R^{b}, —OR^{a}, OC(═O)NR^{a}R^{b}, OC(═O)R^{a} and acyl, wherein R^{a}, R^{b}, R^{c} and R^{d} are each independently selected from the group consisting of H, C₁-C₁₂alkyl, C₁-C₁₂haloalkyl, C₂-C₁₂alkenyl, C₂-C₁₂alkynyl, C₂-Cioheteroalkyl, C₃-C₁₂cycloalkyl, C₃-C₁₂cycloalkenyl, C₂-C₁₂heterocycloalkyl, C₂-C₁₂ heterocycloalkenyl, C₆-C₁₈aryl, C₁-C₁₈heteroaryl, and acyl, or any two or more of R^{a}, R^{b}, R^{c} and R^{d}, when taken together with the atoms to which they are attached form a heterocyclic ring system with 3 to 12 ring atoms.

In some embodiments, each optional substituent is independently selected from the group consisting of: halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, hydroxy, hydroxyalkyl, alkyloxy, alkyloxyalkyl, alkyloxyaryl, alkyloxyheteroaryl, alkenyloxy, alkynyloxy, cycloalkyloxy, cycloalkenyloxy, heterocycloalkyloxy, heterocycloalkenyloxy, aryloxy, heteroaryloxy, arylalkyl, heteroarylalkyl, arylalkyloxy, amino, alkylamino, acylamino, aminoalkyl, arylamino, sulfonyl, alkylsulfonyl, arylsulfonyl, aminosulfonyl, aminoalkyl, —COOH, —SH, and acyl.

Examples of particularly suitable optional substituents include F, Cl, Br, I, CH₃, CH₂CH₃, OH, OCH₃, CF₃, OCF₃, NO₂, NH₂, COOH, COOCH₃ and CN.

As used herein, the term “alkyl” refers to a group or part of a group that is a straight or branched aliphatic hydrocarbon group, preferably a C₁-C₁₂ alkyl, more preferably a C₁-C₁₀ alkyl, most preferably C₁-C₆ unless otherwise noted. Examples of suitable straight and branched C₁-C₆ alkyl substituents include methyl, ethyl, n-propyl, 2-propyl, n-butyl, sec-butyl, t-butyl, hexyl, and the like.

As used herein, the term “halogen” represents chlorine, fluorine, bromine or iodine.

As used herein, the term “heteroatom” refers to a nitrogen (N), oxygen (O) or sulfur (S) atom.

As used herein, the term “alkylene” refers to a bivalent straight or branched chain aliphatic hydrocarbon group. For example, a C₂-C₁₆ alkylene group is a bivalent hydrocarbon group with 2 to 16 carbon atoms in the chain.

As used herein, the term “amine” refers to a —NH₂ or —NH— group, where the valency of the group depends on the surrounding atoms. For example, where an amine group replaces an alkylene unit, the amine group will be an —NH— group. Where the amine group is in a terminal position, the amine group will be a —NH₂ group. One or more hydrogen atoms (where appropriate) may be replaced with non-hydrogen groups, which will result in a substituted amine.

As used herein, the term “amide” refers to a —NH—C(O)— group. It will be understood that the amide group may be present in either the forward or reverse directions and the reference to an amide group encompasses both versions.

As used herein, the term “isomer” refers to and includes all stereoisomers of the compounds of the present invention. Examples of isomers include diastereomers and enantiomers, where appropriate.

As used herein, the term “amino acid” refers to a molecule which contains both an amino and a carboxyl functional group. The amino acid may be a natural or unnatural amino and may also be in equilibrium with its zwitterionic form. The amino acid may contain modifications at either the amino and/or carboxyl terminus, or may contain a free amino group or carboxyl group. Further modification of the amino acid side chain or additional substitutions at other parts of the amino acid are also contemplated.

As used herein, naturally occurring amino acids are the L- or D-form of the twenty amino acids commonly found in nature. These are glycine (Gly, G), alanine (Ala, A), valine (Val, V), leucine (Leu, L), isoleucine (Ile, I), methionine (Met, M), proline (Pro, P), phenylalanine (Phe, F), tryptophan (Trp, W), serine (Ser, S), threonine (Thr, T), asparagine (Asn, N), glutamine (Gln, Q), tyrosine (Tyr, Y), cysteine (Cys, C), lysine (Lys, K), arginine (Arg, R), histidine (His, H), aspartic acid (Asp, D), and glutamic acid (Glu, E).

In certain embodiments of the compounds of Formula (I), X and X¹ may be the same or different and are selected from O, S, NH or N—(C₁₋₁₂alkyl). In some embodiments, X and X¹ are the same. In some embodiments, X and X¹ are the same and are O. In other embodiments, In some embodiments, X and X¹ are the same and are NH. In yet other embodiments, In some embodiments, X and X¹ are the same and are N—(C₁₋₁₂alkyl). In some embodiments, In some embodiments, X and X¹ are the same and are N—C₁alkyl or N-Me.

The linkers in the compounds of Formula (I) are selected from the group consisting of:

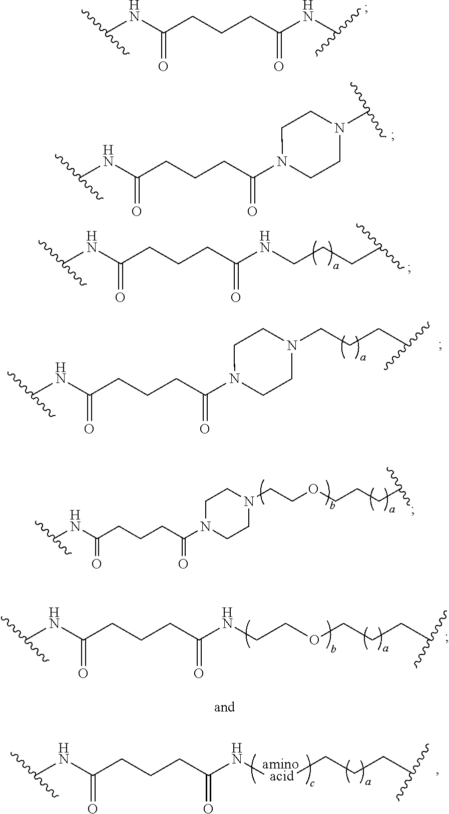

wherein a, b and c are integers independently selected from 1 to 10.

In some embodiments, the linker in the compound of Formula (I) has the following structure:

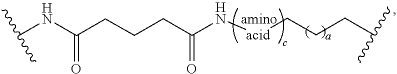

wherein a and c are integers independently selected from 1 to 10 and the one or more amino acids are phenylalanine.

In certain embodiments of the present invention, the linker in the compound of Formula (I) may comprise one or more ethylene oxide groups. In some embodiments, the linker may comprise between 1 and 10 ethylene oxide groups. In some embodiments, the linker may comprise 1, 2, 3, 4, 5, 6, 7, 8, 9 or 10 ethylene oxide groups. In particular embodiments, the linker may comprise 1, 2, 3, 4 or 5 ethylene oxide groups.

In other embodiments of the present invention, the linker in the compound of Formula (I) may comprise one or more amino acids, where the amino acids may be the same or different. In some embodiments, the linker may comprise between 1 and 10 amino acids. In certain embodiments, the linker may comprise 1, 2, 3, 4, 5, 6, 7, 8, 9 or 10 amino acids, where the amino acids are the same or different. In certain embodiments, the linker may comprise 1, 2, 3, 4 or 5 amino acid groups, where the amino acids are the same or different. In some embodiments, the linker may comprise more than one amino acid, where the amino acids present are the same. In other embodiments, the linker may comprise more than one amino acid, where the amino acids present are different.

In some embodiments of the present invention, the linker in the compound of Formula (I) may comprise a piperazine group, where one of the nitrogen atoms of the piperazine group is the nitrogen atom of the propylamide linker:

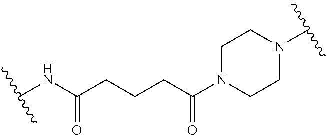 [SMILES: *NC(=O)CCCC(=O)N1CCN(*)CC1]

In the compounds of Formula (I), the variable R represents a terminal group of the sarcophagine. In accordance with the present invention, R is a group selected from the group consisting of H, OH, halogen, cyano, NO₂, NH₂, optionally substituted C₁-C₁₂ alkyl, optionally substituted amino, optionally substituted amide, optionally substituted aryl and a group having the following structure:

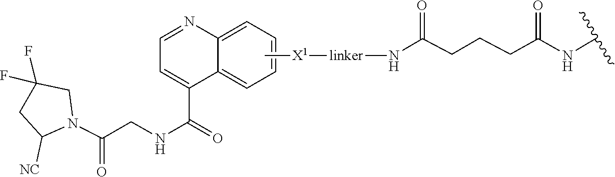

wherein:
X¹ is selected from the group consisting of O, S, NH and N—(C₁₋₁₂alkyl).

In certain embodiments, R is an optionally substituted C₁-C₁₂ alkyl group. In some embodiments, R is an unsubstituted C₁-C₁₂ alkyl group. In other embodiments, R is a substituted C₁-C₁₂ alkyl group that is substituted by one or more groups selected from the group consisting of ═O, ═S, —CN, —NO₂, —CF₃, —OCF₃, alkyl, haloalkyl, haloalkenyl, cycloalkyl, heterocycloalkyl, aryl, heteroaryl, hydroxy, hydroxyalkyl, alkyloxy, alkyloxyalkyl, aryloxy, heteroaryloxy, arylalkyl, heteroarylalkyl, amino, alkylamino, acylamino, aminoalkyl, arylamino, sulfonyl, alkylsulfonyl, arylsulfonyl, aminosulfonyl, aminoalkyl, —COOH, —SH, and acyl. In certain embodiments, R is a C₁-C₁₂ group substituted by an alkyl group. In a specific embodiment, R is an unsubstituted C₁ group.

In certain embodiments of compounds of Formula (I), R is a group having the following structure:

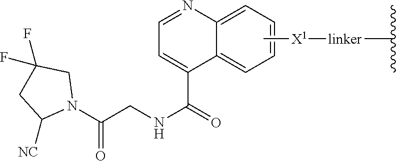

such that the compound of Formula (I) has the structure of a compound of Formula (Ia):

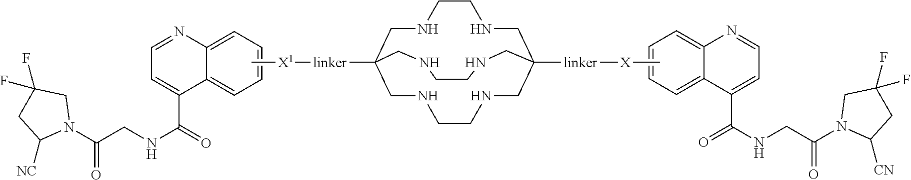

wherein X and X¹ may be the same or different and are selected from the group consisting of O, S, NH and N—(C₁₋₁₂alkyl).

Compounds of Formula (Ia) comprise two moieties that are capable of binding to FAP, where each moiety is linked to a sarcophagine via a linker. Where the linkers and the groups X and X¹ are the same, the compound of Formula (Ia) is symmetrical. Compounds of Formula (Ia) may be considered a “dimeric” form of compounds of Formula (I). Without wishing to be bound by theory, the present inventors believe that the inclusion of a second group that is capable of binding to the target site (i.e. FAP), the compounds of Formula (Ia) may show advantages such as better binding and retention in vivo, which then leads to more efficient with potentially fewer side effects since less of the compound (and the accompanying radioisotope) is administered. The present inventors also believe that the compounds of Formula (Ia) comprising a second moiety capable of binding to FAP is of a suitable size such that the compound is appropriately metabolised.

In certain embodiments, the compound of Formula (I) has one of the following structures:

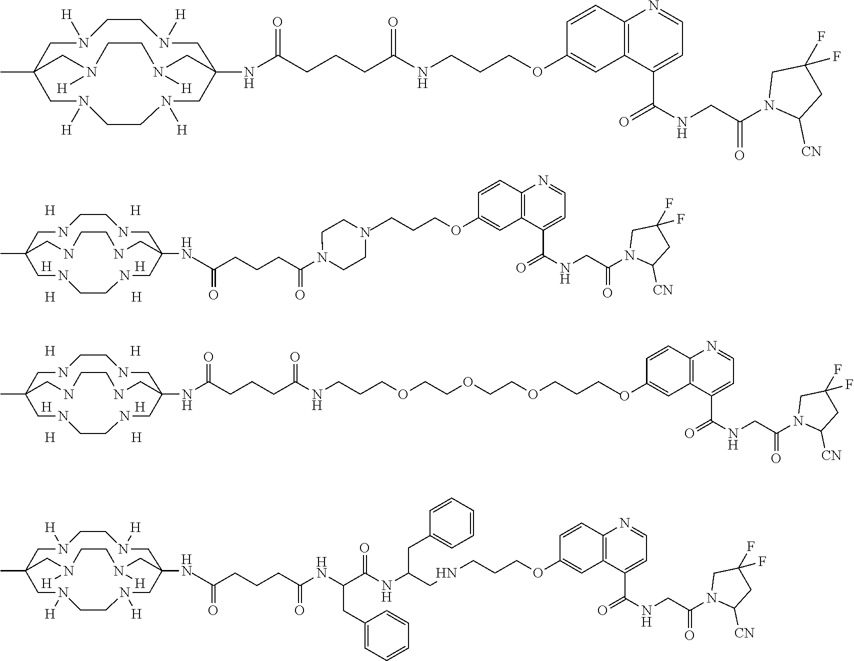

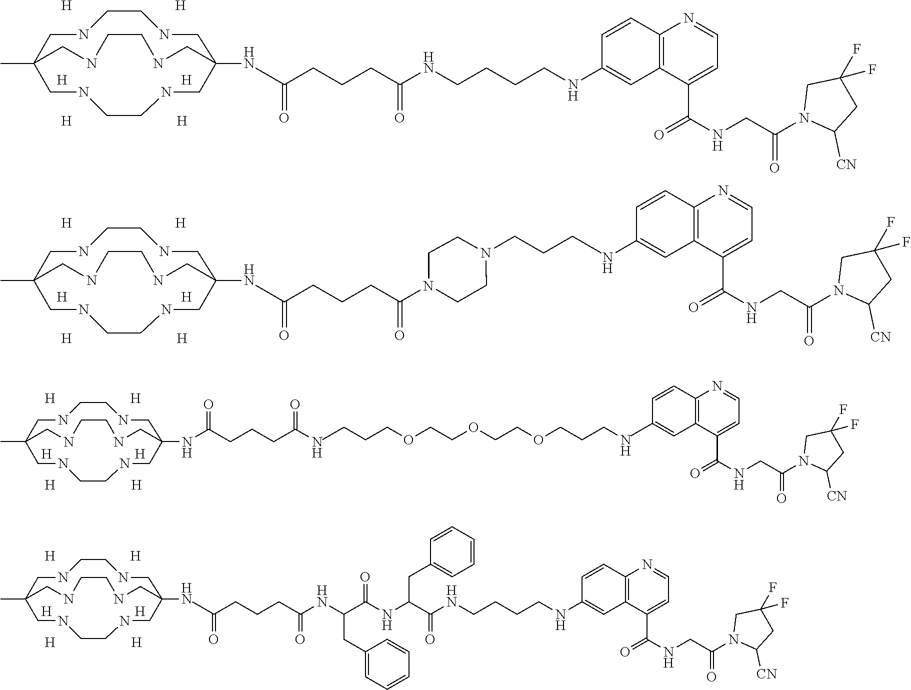

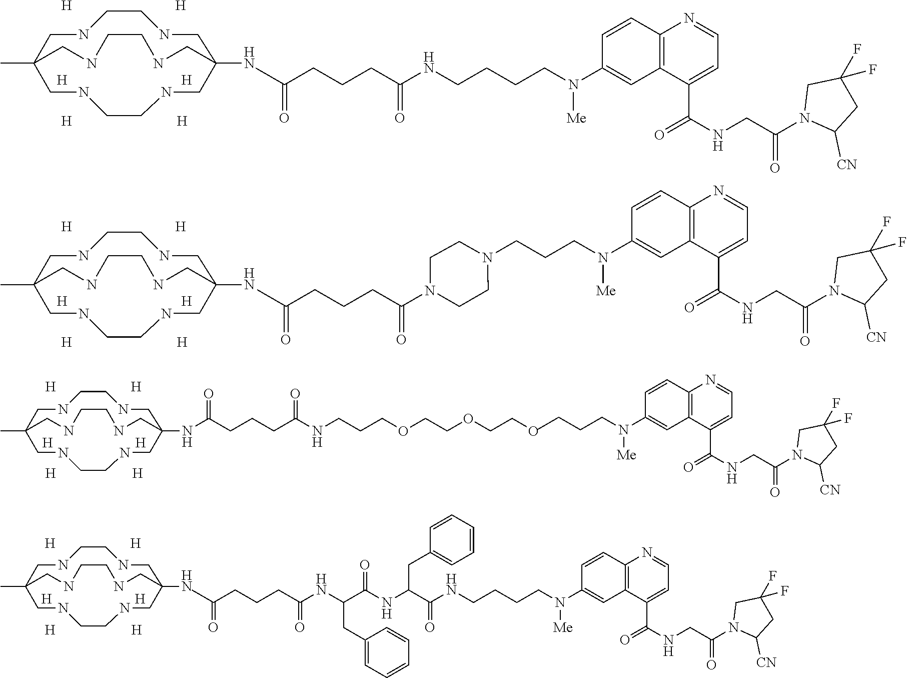

The compounds of Formula (I) have one or more stereocentres. In some embodiments, of the compounds of Formula (I), a particular configuration at a stereocentre is preferred in order to impart the desired biological effect and/or binding properties. For example, the compounds of Formula (I) comprise a nitrile group attached to a carbon atom of the pyrrolidine ring. In a preferred embodiment, the pyrrolidine ring has the configuration as depicted below. The compounds of Formula (I) may also comprise one or more amino acids, where each amino acid has a defined stereochemistry, for example, the stereochemistry in which the amino acid naturally exists. In preferred embodiments, the compound of Formula (I) has one of the following structures:

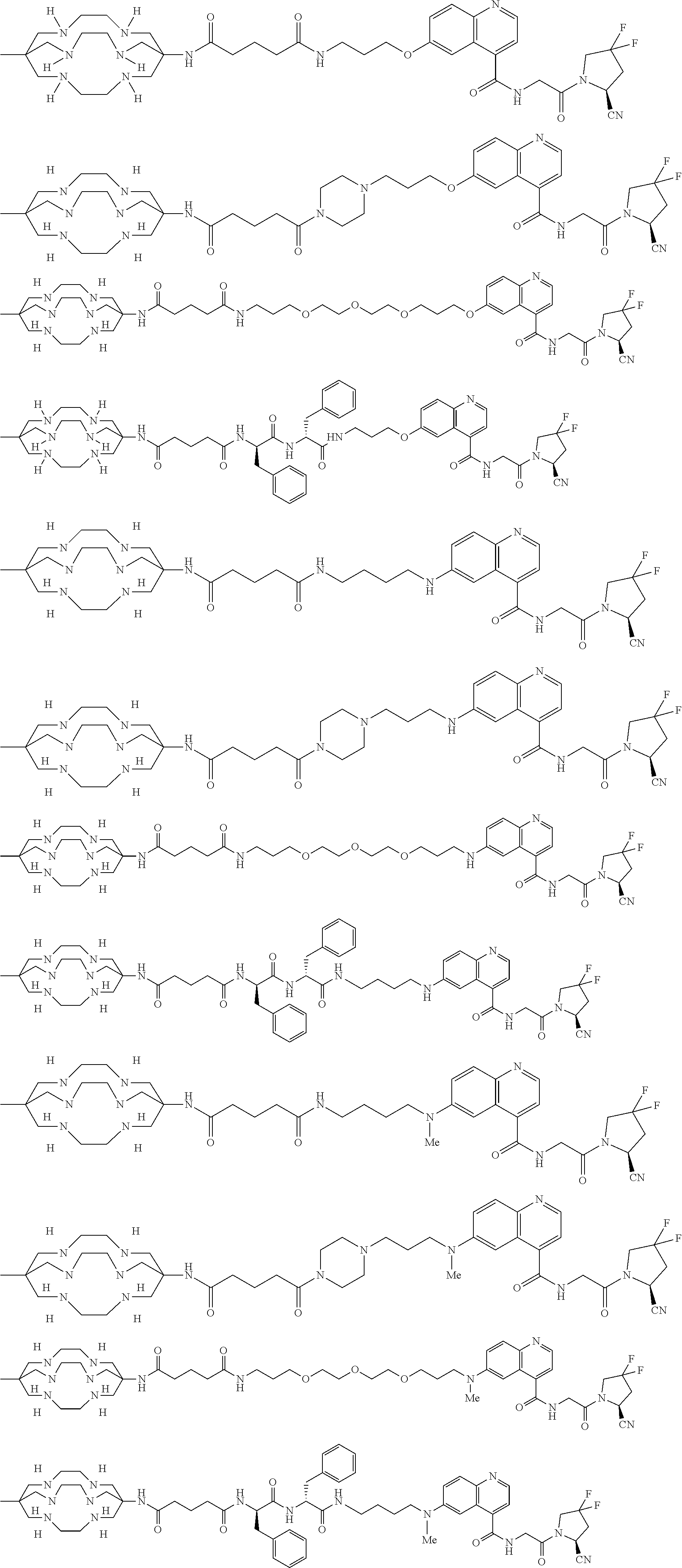

In certain embodiments, the compound of Formula (I) is Sar-FAPi and has the following structure:

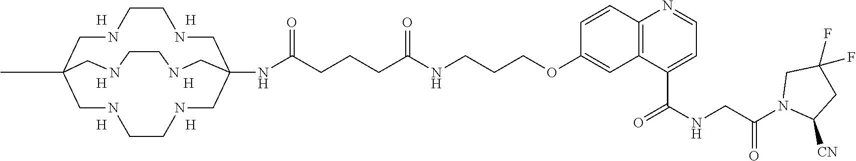

In certain embodiments, the compound of Formula (I) has the structure of Formula (Ia):

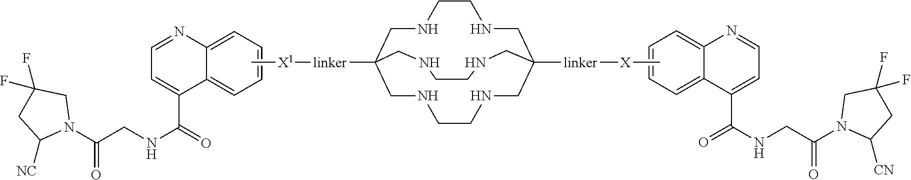

wherein
X and X¹ may be the same or different and are independently selected from the group consisting of O, S, NH and N—(C₁₋₁₂alkyl); and
the linker comprises one or more moieties selected from the group consisting of:

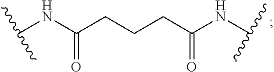 [SMILES: *NC(=O)CCCC(=O)N*]

optionally substituted C₁-C₁₂ alkylene, wherein one or more alkylene groups may be replaced with O, S, NH or N—(C₁₋₁₂alkyl);
one or more amino acids;

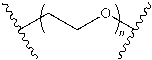

wherein n is an integer from 1 to 10; and

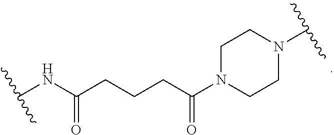 [SMILES: *NC(=O)CCCC(=O)N1CCN(*)CC1]

In certain embodiments, the compound of Formula (Ia) has one of the following structures:

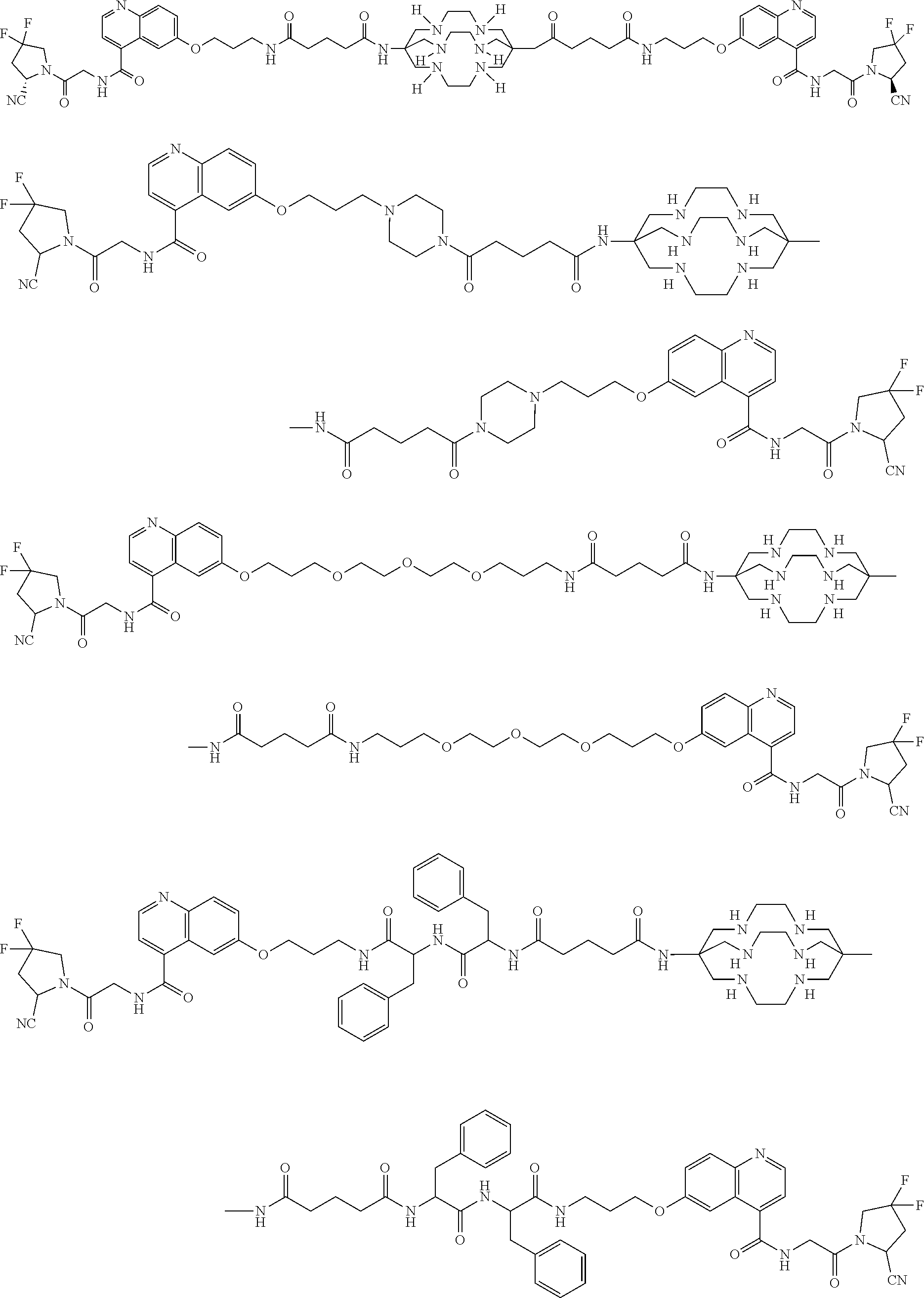

In certain embodiments, the compound of Formula (a) is Sar-bisFAPi and has the following structure:

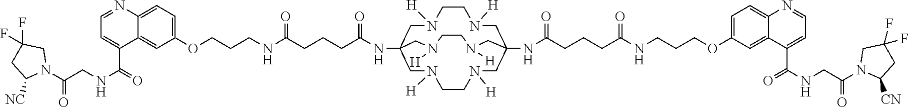 [SMILES: N#C[C@@H]1CC(F)(F)CN1C(=O)CNC(=O)c1ccnc2ccc(OCCCNC(=O)CCCC(=O)NC34CNCCNCC(NC(=O)CCCC(=O)NCCCOc5ccc6nccc(C(=O)NCC(=O)N7CC(F)(F)C[C@H]7C#N)c6c5)(CNCCNC3)CNCCNC4)cc12]

The compounds of Formula (I) may be coordinated with a metal ion via the nitrogen-containing macrocycle to form the corresponding complexes of Formula (I). In an embodiment, the compound of Formula (I) is coordinated with a metal ion.

In an embodiment, the metal ion is an ion of Cu, Tc, Gd, Ga, In, Co, Re, Fe, Mg, Ag, Rh, Pt, Cr, Ni, V, Ir, Zn, Cd, Mn, Ru, Pd, Hg, Ti, Lu, Sc, Zr or Pb.

The present compounds have been found to be particularly useful in binding copper ions. In some embodiments, the metal ion is a radioisotope selected from the group consisting of ⁶⁰Cu, ⁶¹Cu, ⁶²Cu, ⁶⁴Cu and ⁶⁷Cu. In some embodiments, the radioisotope is ⁶⁰Cu. In some embodiments, the radioisotope is ⁶¹Cu. In some embodiments, the radioisotope is ⁶²Cu. In some embodiments, the radioisotope is ⁶⁴Cu. In some embodiments, the radioisotope is ⁶⁷Cu.

The complexes as described herein are radiolabelled with a radioisotope that undergoes spontaneous decay, where these byproducts of decay are detected by various means, such as positron emission tomography (PET) or single-photon emission computed tomography (SPECT). The quality of the images obtained, and subsequently the confidence in any diagnosis based on these images, depend on the ability of the radiolabelled complex that specifically binds to FAP.

Where the metal ion is a radioisotope and the compound for Formula (I) is radiolabelled to form a complex, the complex may be administered for the purposes of radiotherapy or radioimaging. The compounds (and subsequently, the radiolabelled complexes) of Formula (I) contain a group that is capable of binding a biological receptor, therefore the radiolabelled complexes of Formula (I) may be used for the radiotherapy or radioimaging of cancers that are associated with overexpression of the target site to which the compound of Formula (I) binds.

The present inventors have found that the compounds and complexes of Formula (I) containing a sarcophagine and one or more biologically active moieties bound together by linker groups are capable of binding and inhibiting FAP. The combination of each of these components in the compound of Formula (I) allow for administration of the corresponding complex containing a radionuclide, maintaining stability of the complex in vivo and accumulation of the complex at the intended target, i.e. FAP. In order for the biologically active moieties to bind to the intended target and deliver the coordinated radionuclide, there must be a sufficient distance between them in order to prevent any reaction between the groups. The present inventors have found that the linker groups defined herein (i.e. at least the propylamide linker) and the sarcophagine itself provides a compound where the distance between the albumin-binding group and the group capable of binding the biological receptor prevents any such reaction. In addition to contributing to the requisite distance, the linker comprising the nature of the linker group modifies the lipophilicity of the compound, which may in turn improves the hydrolytic stability of the compound and its various fragments.

The compounds of the present invention and complexes thereof with a radioisotope may be used in methods of radioimaging, diagnosis or treatment. In some embodiments, the compounds of the present invention complexed with a radionuclide may be used in a method for radioimaging, diagnosis or treatment of a cancer.

As used herein the terms “treating”, “treatment”, “preventing”, “prevention” and grammatical equivalents refer to any and all uses which remedy the stated neuroendocrine tumour, prevent, retard or delay the establishment of the disease, or otherwise prevent, hinder, retard, or reverse the progression of the disease. Thus the terms “treating” and “preventing” and the like are to be considered in their broadest context. For example, treatment does not necessarily imply that a patient is treated until total recovery. Where the disease displays or a characterized by multiple symptoms, the treatment or prevention need not necessarily remedy, prevent, hinder, retard, or reverse all of said symptoms, but may prevent, hinder, retard, or reverse one or more of said symptoms.

As used herein, the term “cancer” broadly encompasses neoplastic diseases characterised by abnormal cell growth with the potential to invade or spread to other parts of the body. The cancer may be benign, which does not spread to other parts of the body. The cancer may be malignant, meaning that the cancer cells can spread through the circulatory system or lymphatic system. The term as used herein includes all malignant, i.e. cancerous, disease states. The cancer may be present as a tumour. In certain embodiments, the term cancer as used herein refers to a cancer that is characterised by overexpression of FAP.

As used herein, the term “tumour” refers to any malignant cancerous or pre-cancerous cell growths. The term may also include leukemias, but is particularly directed to solid tumours or carcinomas.

Radioimaging of a cancer of associated with the expression of a receptor in connection with the administration of a complex of Formula (I) also relies upon the selection of a suitable radionuclide. For example, where the intended use of a complex of Formula (I) is for the purposes of radioimaging, the selected radionuclide should have a sufficiently long half-life such that detection of radionuclide decay allows for images of a sufficient quality to be obtained. This also requires that the compound of Formula (I) itself, i.e. the ligand coordinating the radionuclide, be sufficiently stable with respect to radioactive decay. The present inventors have found that decomposition of a complex of Formula (I) by radiolysis (i.e. as a result of the radioactivity of the radionuclide) is minimized and that the complex of Formula (I) generally remains intact in this regard.

Radioimaging of a subject to which a radiolabeled compound of Formula (I) is administered may be by positron emission tomography (PET) or by single-photon emission computed tomography (SPECT). In an embodiment, the present invention provides a method for radioimaging a subject in need thereof, the method comprising administering a compound of Formula (I) complexed with a radionuclide. In an embodiment, the method comprises administering a compound of Formula (I) complexed with a copper radionuclide. In another embodiment, the method comprises administering a compound of Formula (I) complexed with ⁶⁴CU.

In an embodiment, radioimaging of the subject after administration of the compound of Formula (I) complexed by a radionuclide is by PET. In another embodiment, radioimaging of the subject after administration of the compound of Formula (I) complexed by a radionuclide is by SPECT.

The term “subject” as used herein refers to mammals and includes humans, primates, livestock animals (e.g. sheep, pigs, cattle, horses, donkeys), laboratory test animals (e.g. mice, rabbits, rats, guinea pigs), performance and show animals (e.g. horses, livestock, dogs, cats), companion animals (e.g. dogs, cats) and captive wild animals. Preferably, the mammal is human or a laboratory test animal. Even more preferably, the mammal is a human.

The compounds of the present invention complexed with a radionuclide may be administered to a subject in need thereof as a composition by a parenteral route. Administration by intravenous injection may be preferred. Alternatively, the formulations of the present invention may be given by intraarterial or other routes, for delivery into the systemic circulation. The subject to which the compound is administered is then placed into a PET (or SPECT) scanner and images showing the localisation of the complex, and subsequently location of any cancers or tumours, are obtained. This then allows for diagnosis and detection of a cancer or tumour.

The compounds of the present invention and complexes thereof with a radionuclide may be used in methods of treatment of diseases, such as cancers. When complexed with a suitable radionuclide, the complexes of the present invention may be administered to a subject in need thereof. The methods disclosed herein comprise administration of a therapeutically effective amount of a radiolabeled compound of the present invention to a subject in need thereof. In an embodiment, the present invention provides a method for treating a disease in a subject in need thereof, the method comprising administering a therapeutically effective amount of compound of Formula (I) complexed with a radionuclide.

The term “therapeutically effective amount” or “effective amount” is an amount sufficient to effect beneficial or desired clinical results. An effective amount can be administered in one or more administrations. For the purposes of radioimaging, an effective amount is sufficient for an image showing the localisation of the compound of Formula (I) administered to the subject, owing to the detection of the products of decay from the radioisotope that is complexed with the compound. For the purposes of treatment, an effective amount is typically sufficient to palliate, ameliorate, stabilize, reverse, slow and/or delay the progression of the cancer.

In an embodiment, the present invention provides a method for treating a cancer, the method comprising administering a compound of Formula (I) complexed with a radionuclide. In an embodiment, the method comprises administering a compound of Formula (I) complexed with a copper radionuclide. In another embodiment, the method comprises administering a compound of Formula (I) complexed with ⁶⁷Cu. In certain embodiments, the method comprises administering a compound of Formula (I) complexed with ⁶⁷Cu, wherein the cancer is associated with fibroblast activation protein (FAP). In another embodiment, the method comprises administering a compound of Formula (I), which comprises a biologically active moiety that binds to FAP. In another embodiment, the method comprises administering a compound of Formula (I), which comprises a biologically active moiety that inhibits FAP. In another embodiment, the method comprises administering a compound of Formula (I), which comprises a biologically active moiety that binds to and inhibits FAP.

Examples of types of cancers that may be treated by administration of compounds of the present invention include those in the categories of carcinoma, lymphoma and sarcoma. Examples of carcinomas include, but are not limited to, adenocarcinoma, acinic cell adenocarcinoma, adrenal cortical carcinomas, alveoli cell carcinoma, anaplastic carcinoma, basaloid carcinoma, basal cell carcinoma, bronchiolar carcinoma, bronchogenic carcinoma, renaladinol carcinoma, embryonal carcinoma, anometroid carcinoma, fibrolamolar liver cell carcinoma, follicular carcinomas, giant cell carcinomas, hepatocellular carcinoma, intraepidermal carcinoma, intraepithelial carcinoma, leptomanigio carcinoma, medullary carcinoma, melanotic carcinoma, menigual carcinoma, mesometonephric carcinoma, oat cell carcinoma, squamal cell carcinoma, sweat gland carcinoma, transitional cell carcinoma, and tubular cell carcinoma. Examples of sarcomas include, but are not limited to, amelioblastic sarcoma, angiolithic sarcoma, botryoid sarcoma, endometrial stroma sarcoma, ewing sarcoma, fascicular sarcoma, giant cell sarcoma, granulositic sarcoma, immunoblastic sarcoma, juxaccordial osteogenic sarcoma, coppices sarcoma, leukocytic sarcoma (leukemia), lymphatic sarcoma (lymphosarcoma), medullary sarcoma, myeloid sarcoma (granulocytic sarcoma), austiogenci sarcoma, periosteal sarcoma, reticulum cell sarcoma (histiocytic lymphoma), round cell sarcoma, spindle cell sarcoma, synovial sarcoma, and telangiectatic audiogenic sarcoma. Examples of lymphomas include, but are not limited to, Hodgkin's disease and lymphocytic lymphomas, such as Burkitt's lymphoma, nodular poorly differentiated lymphocytic lymphoma, nodular mixed lymphoma, nodular histocytic lymphoma and diffuse lymphoma.

Examples of cancers that may be treated using the compounds disclosed herein include, but are not limited to, Hodgkin's disease, non-Hodgkin's lymphomas, acute lymphocytic leukemia, multiple myeloma, breast carcinomas, ovarian carcinomas, lung carcinomas, Wilms' tumour, testicular carcinomas, soft-tissue sarcomas, chronic lymphocytic leukemia, primary macroglobulinemia, bladder carcinomas, chronic granulocytic leukemia, primary brain carcinomas, malignant melanoma, small-cell lung carcinomas, stomach carcinomas, colon carcinomas, malignant pancreatic insulinoma, malignant carcinoid carcinomas, malignant melanomas, choriocarcinomas, mycosis fungoides, head and neck carcinomas, osteogenic sarcoma, pancreatic carcinomas, acute granulocytic leukemia, hairy cell leukemia, rhabdomyosarcoma, Kaposi's sarcoma, genitourinary carcinomas, thyroid carcinomas, esophageal carcinomas, malignant hypercalcemia, renal cell carcinomas, endometrial carcinomas, polycythemia vera, essential thrombocytosis, adrenal cortex carcinomas, skin cancer, and prostatic carcinomas.

In certain embodiments, the cancer is lung cancer, testicular cancer, renal cancer, bladder cancer, kidney or renal cancer, ovarian cancer, breast cancer, fallopian tube cancer, uterine leiomyoma, prostate cancer, non-Hodgkin's lymphoma, colon cancer, lipoma, basal cell skin carcinoma, squamous cell skin carcinoma, osteosarcoma, acute myelogenous leukemia (AML), pancreatic cancer, prostate cancer, CNS cancer, retinoblastoma, neuroblastoma, glioblastoma, Kaposi's sarcoma, Ewing's sarcoma, rhabdomyosarcoma, hermangioma, a solid tumour, a blood-borne tumour, leukemia or melanoma.

Examples of other specific types of cancers include lung cancers (e.g. bronchogenic carcinoma, small cell lung cancer (SCLC), non-small cell lung cancer (NSCLC), adenocarcinoma of the lung), kidney cancer (e.g. nephroblastoma or Wilms' tumour, renal cell carcinoma) acoustic neuroma, adenocarcinoma, adrenal gland cancer, anal cancer, angiosarcoma (e.g. lymphangiosarcoma, lymphangioendotheliosarcoma, hemangiosarcoma), appendix cancer, benign monoclonal gammopathy, biliary cancer (e.g. cholangiocarcinoma), bladder cancer, breast cancer (e.g. adenocarcinoma of the breast, papillary carcinoma of the breast, mammary cancer, medullary carcinoma of the breast), brain cancer (e.g. meningioma, glioblastomas, glioma (e.g. astrocytoma, oligodendroglioma), medulloblastoma), bronchus cancer, carcinoid tumour, cervical cancer (e.g. cervical adenocarcinoma), choriocarcinoma, chordoma, craniopharyngioma, colorectal cancer (e.g. colon cancer, rectal cancer, colorectal adenocarcinoma), connective tissue cancer, epithelial carcinoma, ependymoma, endotheliosarcoma (e.g. Kaposi's sarcoma, multiple idiopathic hemorrhagic sarcoma), endometrial cancer (e.g. uterine cancer, uterine sarcoma), esophageal cancer (e.g. adenocarcinoma of the esophagus, Barrett's adenocarcinoma), Ewing's sarcoma, ocular cancer (e.g. intraocular melanoma, retinoblastoma), familiar hypereosinophilia, gall bladder cancer, gastric cancer (e.g. stomach adenocarcinoma), gastroesophageal cancer, gastrointestinal stromal tumour (GIST), germ cell cancer, head and neck cancer (e.g. head and neck squamous cell carcinoma, oral cancer (e.g. oral squamous cell carcinoma), throat cancer (e.g. laryngeal cancer, pharyngeal cancer, nasopharyngeal cancer, oropharyngeal cancer)), heavy chain disease (e.g. alpha chain disease, gamma chain disease, mu chain disease, hemangioblastoma, hypopharynx cancer, inflammatory myofibroblastic tumours, immunocytic amyloidosis), liver cancer (e.g. hepatocellular cancer (HCC), malignant hepatoma, hepatobiliary cancer), leiomyosarcoma (LMS), mastocytosis (e.g. systemic mastocytosis), muscle cancer, myelodysplastic syndrome (MDS), mesothelioma, myeloproliferative disorder (MPD) (e.g. polycythemia vera (PV), essential thrombocytosis (ET), agnogenic myeloid metaplasia (AMM) or myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g. neurofibromatosis (NF) type 1 or type 2, schwannomatosis), neuroendocrine cancer (e.g. gastroenteropancreatic neuroendoctrine tumour (GEP-NET), carcinoid tumour), osteosarcoma (e.g. bone cancer), ovarian cancer (e.g. cystadenocarcinoma, ovarian embryonal carcinoma, ovarian adenocarcinoma), papillary adenocarcinoma, pancreatic cancer (e.g. pancreatic adenocarcinoma, intraductal papillary mucinous neoplasm (IPMN), Islet cell tumours), penile cancer (e.g. Paget's disease of the penis and scrotum), pinealoma, primitive neuroectodermal tumour (PNT), plasma cell neoplasia, paraneoplastic syndromes, intraepithelial neoplasm, rectal cancer, rhabdomyosarcoma, salivary gland cancer, skin cancer (e.g. squamous cell carcinoma (SCC), keratoacanthoma (KA), melanoma, basal cell carcinoma (BCC)), small bowel cancer (e.g. appendix cancer), soft tissue sarcoma (e.g. malignant fibrous histiocytoma (MFH), liposarcoma, malignant peripheral nerve sheath tumour (MPNST), chondrosarcoma, fibrosarcoma, myxosarcoma), sebaceous gland carcinoma, small intestine cancer, sweat gland carcinoma, synovioma; testicular cancer (e.g. seminoma, testicular embryonal carcinoma), thyroid cancer (e.g. papillary carcinoma of the thyroid, papillary thyroid carcinoma (PTC), medullary thyroid cancer), urethral cancer, vaginal cancer, and vulvar cancer (e.g. Paget's disease of the vulva).

Examples of specific types of breast cancer include lobular carcinoma in situ (LCIS), a ductal carcinoma in situ (DCIS), an invasive ductal carcinoma (IDC), inflammatory breast cancer, Paget disease of the nipple, phyllodes tumour, angiosarcoma, adenoid cystic carcinoma, low-grade adenosquamous carcinoma, medullary carcinoma, mucinous carcinoma, papillary carcinoma, tubular carcinoma, metaplastic carcinoma, micropapillary carcinoma, mixed carcinoma, or another breast cancer, including triple negative (TNBC), HER positive, neoadjuvant HER2 negative, estrogen receptor positive, progesterone receptor positive, HER and estrogen receptor positive, HER and progesterone receptor positive, estrogen and progesterone receptor positive, and HER and estrogen and progesterone receptor positive.

Examples of specific types of ovarian cancer include epithelial ovarian carcinoma (EOC), maturing teratoma, dysgerminoma, endodermal sinus tumour, granulosa-theca tumours Sertoli-Leydig cell tumour, primary peritoneal carcinoma, small cell carcinoma of the ovary (SCCO), teratoma of the ovary, sex cord-stromal ovarian cancer, dysgerminoma ovarian germ cell cancer, choriocarcinoma, carcinosarcoma, adenosarcoma, leiomyosarcoma, fibrosarcoma, and Krukenberg tumour.

Examples of specific types of pancreatic cancer include tumours affecting the exocrine gland, exocrine tumours, endocrine tumour, islet cell tumours, neurendocrine tumours, cystic tumours, cancer of the acinar cells, insulinoma, somatostatinoma, gastrinoma, glucagonoma, adenocarcinoma of the pancreas, pancreatoblastoma, sarcoma of the pancreas, adenosquamous carcinomas, colloid carcinoma, hepatoid carcinoma, intraductal papillary mucinous neoplasm, mucinous cystic neoplasm, pancreatic intraepithelial neoplasia, pancreatoblastoma, serous cystadenoma, signet ring cell carcinoma, solid-pseudopapillary neoplasm, and undifferentiated carcinoma with osteoclast-like giant cells.

Examples of specific types of prostate cancer include prostate adenocarcinoma, acinar adenocarcinoma, ductal adenocarcinoma, transitional cell (or urothelial) cancer, squamous cell cancer, small cell prostate cancer, carcinoid, sarcoma, small cell carcinoma, neuroendocrine tumour, and transitional cell carcinoma.

In certain embodiments, the cancer is selected from the group consisting of epithelial ovarian cancer, ovarian carcinoma, osteosarcoma, pancreatic adenocarcinoma, colorectal cancer, lung cancer, non-small cell lung cancer, gastric cancer, endometrial carcinoma, pancreatic adenocarcinoma, medullary thyroid carcinoma, differentiated thyroid cancer, breast cancer, invasive ductal carcinoma of the breast, oral squamous cell carcinoma, esophageal cancer, renal cell cancer, insulinoma, prostate cancer, neuroendocrine differentiated prostate cancer, pheochromocytoma, adenoid cystic cancer, hepatocellular carcinoma, cervical cancer, small intestine cancer, neuroendocrine tumour, anal cancer, chordoma, desmoid tumour, head and neck cancer, thymus cancer, pancreatic cancer, cholangiocellular carcinoma, esophageal cancer, salivary gland cancer, sarcoma, carcinoma of unknown primary cancer.

The compounds and complexes of the present invention can be administered alone or in the form of a pharmaceutical composition in combination with a pharmaceutically acceptable carrier, diluent or excipient. The compounds of the invention, while effective themselves, are typically formulated and administered in the form of their pharmaceutically acceptable salts as these forms are typically more stable, more easily crystallised and have increased solubility.

The compounds of the present invention are typically used in the form of pharmaceutical compositions that are formulated depending on the desired mode of administration. The compositions are prepared in manners well known in the art.

In using the compounds of the invention, they can be administered in any form or mode which makes the compound available for the desired application (imaging or radiotherapy). One skilled in the art of preparing formulations of this type can readily select the proper form and mode of administration depending upon the particular characteristics of the compound selected, the condition to be treated, the stage of the condition to be treated and other relevant circumstances. Reference is made to Remington's Pharmaceutical Sciences, 19th edition, Mack Publishing Co. (1995) for further information. In certain embodiments, the compound of Formula (I) is administered by injection. In other embodiments, the compound of Formula (I) is administered intravenously. In other embodiments, the compound of Formula (I) is administered directly to the location of the cancer.

The invention in other embodiments provides a pharmaceutical pack or kit comprising one or more containers filled with one or more of the ingredients of the pharmaceutical compositions of the invention. In such a pack or kit can be found at least one container having a unit dosage of the agent(s). Conveniently, in the kits, single dosages can be provided in sterile vials so that the clinician can employ the vials directly, where the vials will have the desired amount and concentration of compound and radio nucleotide which may be admixed prior to use. Associated with such container(s) can be various written materials such as instructions for use, or a notice in the form prescribed by a governmental agency regulating the manufacture, use or sale of pharmaceuticals, imaging agents or biological products, which notice reflects approval by the agency of manufacture, use or sale for human administration.

In an embodiment, the invention provides compositions comprising a compound as described above together with one or more pharmaceutically acceptable excipients.

Pharmaceutical compositions of this invention for parenteral injection comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin.

If desired, and for more effective distribution, the compounds can be incorporated into slow release or targeted delivery systems such as polymer matrices, liposomes, and microspheres.

The injectable formulations can be sterilized, for example, by filtration through a bacterial-retaining filter, or by incorporating sterilizing agents in the form of sterile solid compositions that can be dissolved or dispersed in sterile water or other sterile injectable medium just prior to use.

The present invention also provides processes for the synthesis or preparation of compounds of the invention. The present inventors have found that established procedures that may be used for the preparation of compounds of the present invention by various coupling procedures and conditions does not allow the desired compounds to be accessed. This is largely due to incompatibilities between functional groups, issues of reagent solubility and general issues of reactivity.

The present inventors have found that the compounds of Formulae (I) and (Ia) may be synthesised according to various routes, for example, according to the schemes disclosed in WO 2019/154886 and WO 2019/154859, the disclosures of which are incorporated herein by reference. A simplified route to access compounds of Formula (I) is shown in Scheme 1, where the isoquinolone (or N-acylated aminoacyl pyrrolidine-substituted isoquinoline) group is coupled sequentially to a linker and the sarcophagine moiety.

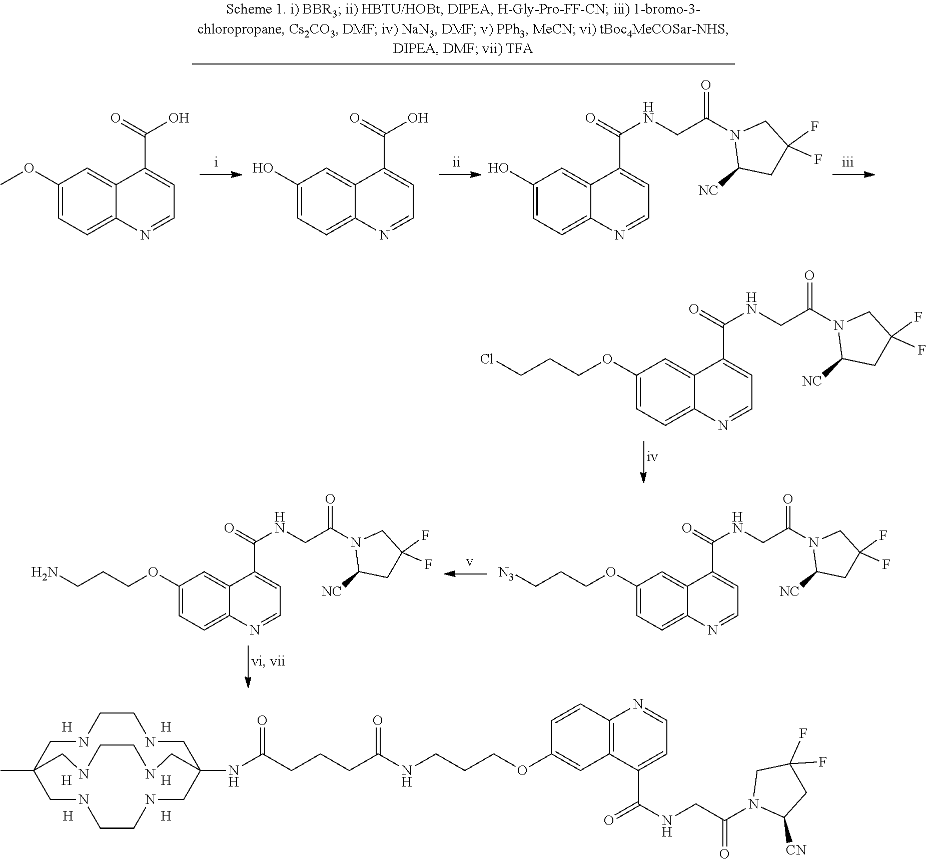

Alternatively, the compounds of Formula (I) the difluoropyrrolidine-substituted isoquinolone may be produced in several steps from simple precursor molecules, as shown in Schemes 2 to 4.

Scheme 2 shows the synthesis of an embodiment of a compound of Formula (I) comprising a specific linker, where the difluoropyrrolidine group is prepared from precursors:

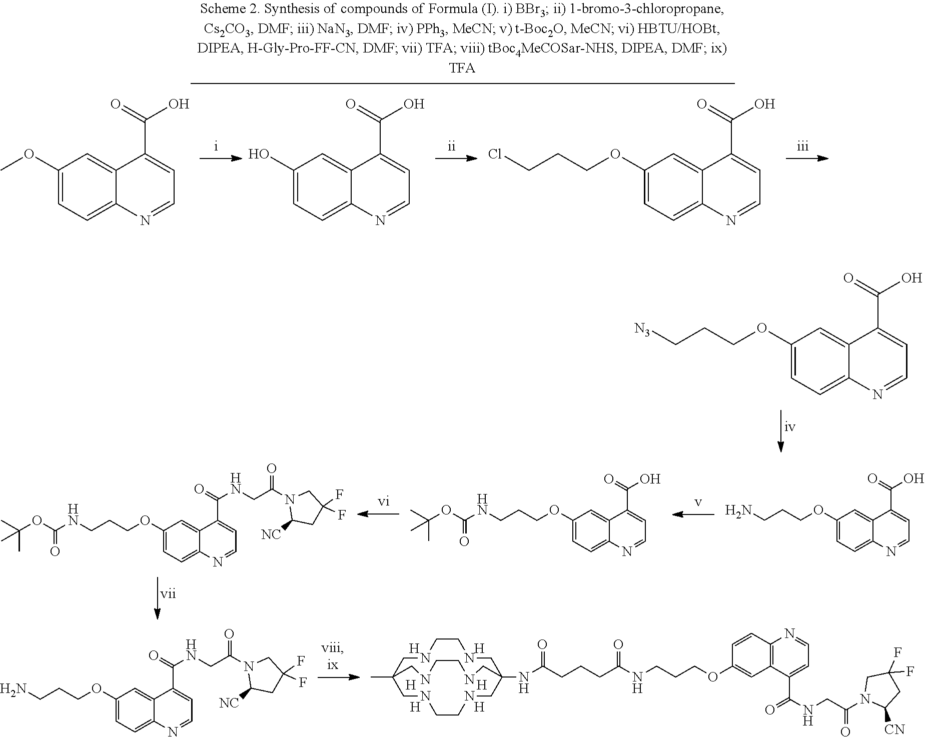

Scheme 3 shows the synthesis of another embodiment of a compound of Formula (I) with a linker comprising one or more amino acids, where the difluoropyrrolidine group is prepared from precursors:

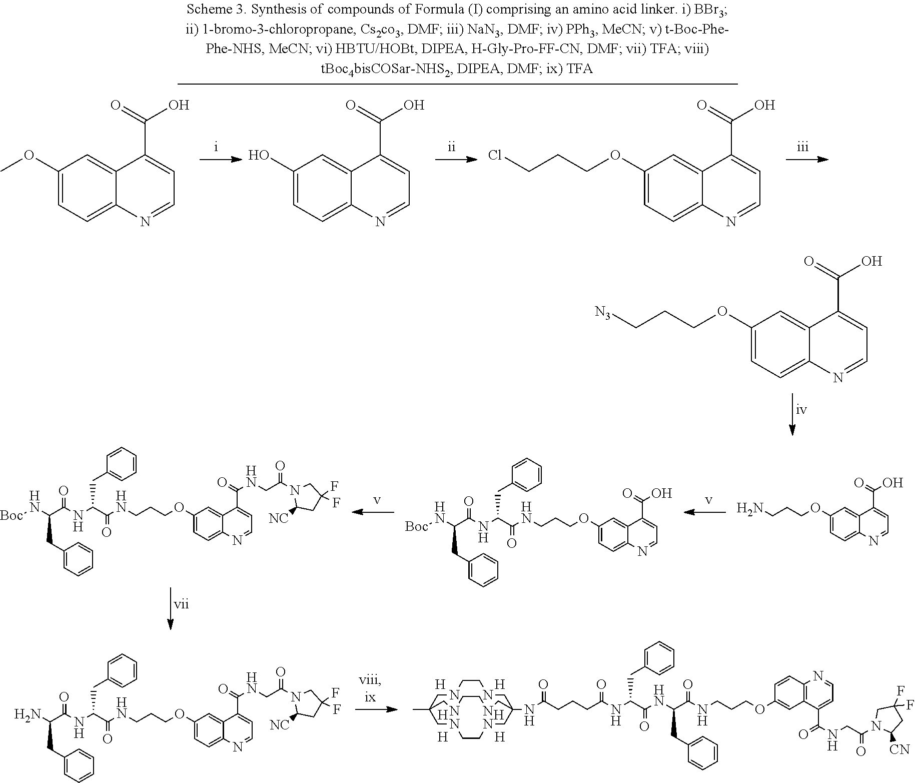

Scheme 4 shows the synthesis of an embodiment of a compound of Formula (Ia) comprising a sarcophagine and two difluoropyrrolidine groups prepared from precursors, wherein each moiety is bound by a linker.

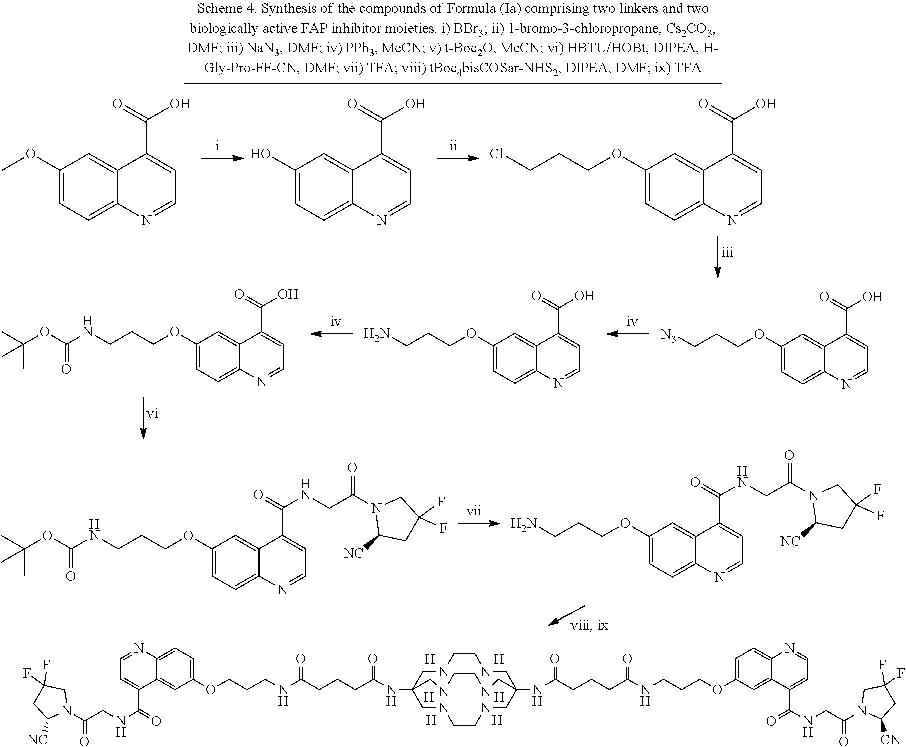

The present inventors have also found that the route described in Schemes 5 and 6 can also provide access to the compounds of Formula (I) or Formula (Ia). In contrast to the routes described in Schemes 1 to 4, the route detailed in Scheme 5 does not use an intermediate azide functionality, which are associated with various health and safety issues. Furthermore, the routes in Schemes 5 and 6 have fewer steps, since fewer conversions of functional groups are required. Without wishing to be bound by theory, the present inventors believe that the synthetic routes disclosed herein provide various methods for accessing compounds of the present invention.

Scheme 5 shows a modified route to compounds of Formula (I), where the component for inhibition of FAP is prepared in 3 steps from a pyridine precursor similar to those used in Schemes 1 to 4. The TFA-protected form of the inhibitory component is then coupled under standard peptide coupling conditions to provide a compound of Formula (I). In Scheme 6, the MeCOSar (i.e. methyl substituted sarcophagine) is replaced with a bisCOSar.

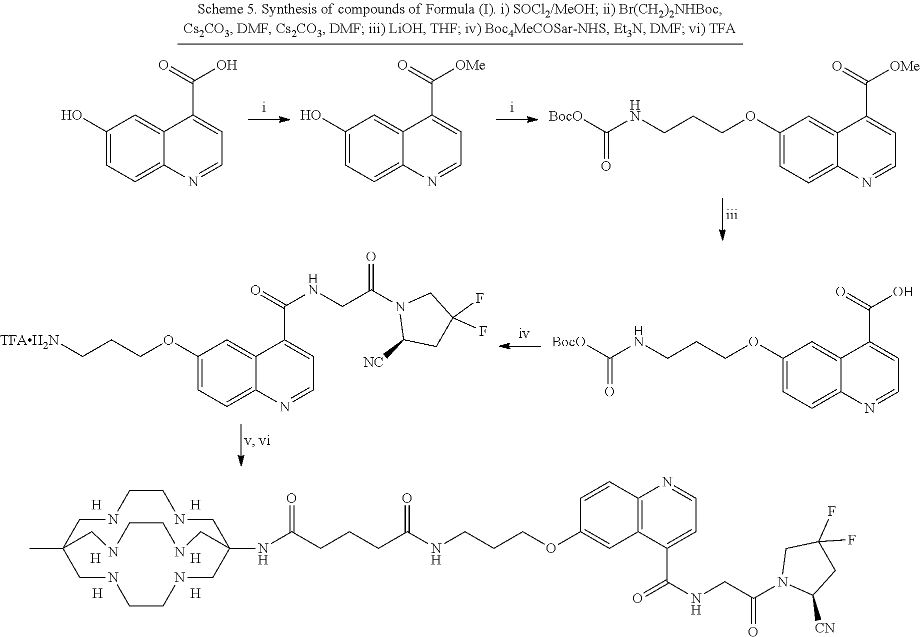

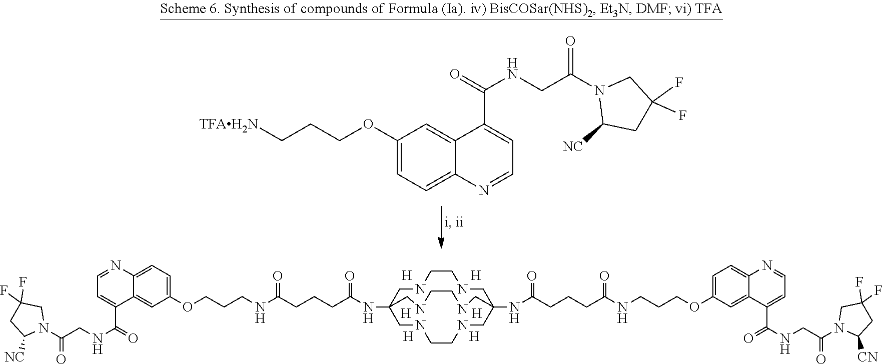

Accordingly, in a further aspect, the present invention provides a method for producing a compound of Formula (I) or a pharmaceutically acceptable salt thereof, the method comprising the step of:
i) preparing a compound of Formula A or a protected form thereof:

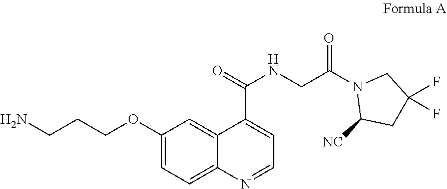 [SMILES: N#C[C@@H]1CC(F)(F)CN1C(=O)CNC(=O)c1ccnc2ccc(OCCCN)cc12]

ii) preparing a compound of Formula B or a protected form thereof:

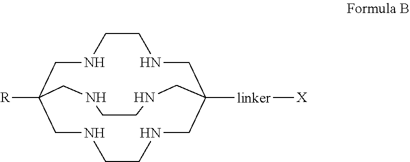

iii) coupling the compound of Formula A or a protected form thereof with the compound of Formula B or a protected form thereof,
   to produce the compound of Formula (I) or a protected form thereof,
   wherein:
R is a group selected from the group consisting of H, OH, halogen, cyano, NO₂, NH₂, optionally substituted C₁-C₁₂ alkyl, optionally substituted amino, optionally substituted amide, optionally substituted aryl and a group having the following structure:

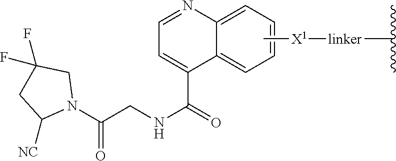

wherein:
X and X¹ may be the same or different and are independently selected from the group consisting of O, S, NH and N—(C₁₋₁₂alkyl).

In an embodiment, the protected form of a compound of Formula A contains a nitrogen protecting group or an oxygen protecting group. In another embodiment, the protected form of a compound of Formula B contains a nitrogen protecting group or an oxygen protecting group.

As used herein, the term “oxygen protecting group” refers to a group that can prevent the oxygen moiety reacting during further derivatisation of the protected compound and which can be readily removed when desired. In one embodiment, the protecting group is removable in the physiological state by natural metabolic processes. Examples of oxygen protecting groups include acyl groups (such as acetyl), ethers (such as methoxy methyl ether (MOM), α-methoxy ethoxy methyl ether (MEM), p-methoxy benzyl ether (PMB), methylthio methyl ether, pivaloyl (Piv), tetrahydropyran (THP)), and silyl ethers (such as trimethylsilyl (TMS) tert-butyl dimethyl silyl (TBDMS) and triisopropylsilyl (TIPS) groups.

As used herein, the term “nitrogen protecting group” refers to a group that can prevent the nitrogen moiety reacting during further derivatisation of the protected compound and which can be readily removed when desired. In one embodiment, the protecting group is removable in the physiological state by natural metabolic processes and in essence the protected compound is acting as a prodrug for the active unprotected species. Examples of suitable nitrogen protecting groups that may be used include formyl, trityl, phthalimido, acetyl, trichloroacetyl, chloroacetyl, bromoacetyl, iodoacetyl; urethane-type blocking groups such as benzyloxycarbonyl (CBz), 4-phenylbenzyloxycarbonyl, 2-methylbenzyloxycarbonyl, 4-methoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, t-butoxycarbonyl (tBoc), 2-(4-xenyl)-isopropoxycarbonyl, 1,1-diphenyleth-1-yloxycarbonyl, 1,1-diphenylprop-1-yloxycarbonyl, 2-phenylprop-2-yloxycarbonyl, 2-(p-toluyl)-prop-2-yloxy-carbonyl, cyclo-pentanyloxy-carbonyl, 1-methylcyclopentanyloxycarbonyl, cyclohexanyloxycarbonyl, 1-methylcyclohexanyloxycarbonyl, 2-methylcyclohexanyloxycarbonyl, 2-(4-toluylsulfono)-ethoxycarbonyl, 2-(methylsulfono)ethoxycarbonyl, 2-(triphenylphosphino)-ethoxycarbonyl, fluorenylmethoxycarbonyl (Fmoc), 2-(trimethylsilyl)ethoxycarbonyl, allyloxycarbonyl, 1-(trimethylsilylmethyl)prop-1-enyloxycarbonyl, 5-benzisoxalylmethoxy carbonyl, 4-acetoxybenzyloxycarbonyl, 2,2,2-trichloroethoxycarbonyl, 2-ethynyl-2-propoxycarbonyl, cyclopropylmethoxycarbonyl, 4-(decyloxy)benzyloxycarbonyl, isobornyloxycarbonyl, 1-piperidyloxycarbonlyl and the like; benzoylmethylsulfono group, 2-nitrophenylsulfenyl, diphenylphosphine oxide, and the like. The actual nitrogen protecting group employed is not critical so long as the derivatised nitrogen group is stable to the condition of subsequent reaction(s) and can be selectively removed as required without substantially disrupting the remainder of the molecule including any other nitrogen protecting group(s). Further examples of these groups are found in: Greene, T. W. and Wuts, P. G. M., Protective Groups in Organic Synthesis, Second edition; Wiley-Interscience: 1991; Chapter 7; McOmie, J. F. W. (ed.), Protective Groups in Organic Chemistry, Plenum Press, 1973; and Kocienski, P. J., Protecting Groups, Second Edition, Thieme Medical Pub., 2000.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

### Examples

The following examples are illustrative of the disclosure and should not be construed as limiting in any way the general nature of the disclosure of the description throughout this specification.

### Synthesis of Compounds of the Invention

The agents of the various embodiments may be prepared using the reaction routes and synthesis schemes as described herein, employing the techniques available in the art using starting materials that are readily available. The preparation of particular compounds of the embodiments is described in detail in the following examples, but the artisan will recognize that the chemical reactions described may be readily adapted to prepare a number of other agents of the various embodiments. For example, the synthesis of non-exemplified compounds may be successfully performed by modifications apparent to those skilled in the art, e.g. by appropriately protecting interfering groups, by changing to other suitable reagents known in the art, or by making routine modifications of reaction conditions. A list of suitable protecting groups in organic synthesis can be found in T. W. Greene's Protective Groups in Organic Synthesis, 3^{rd} Edition, John Wiley & Sons, 1991. Alternatively, other reactions disclosed herein or known in the art will be recognized as having applicability for preparing other compounds of the various embodiments. Reagents useful for synthesizing compounds may be obtained or prepared according to techniques known in the art.

### Instrumentation

Mass spectra were collected using a Thermo Scientific Exactive Plus OrbiTrap LC/MS (Thermo Fisher Scientific, Massachusetts, USA) and calibrated to internal references.

Copper-64 was supplied from the University of Queensland as a solution in HCl (0.1 M). Copper was used approx. 48 hours (4 half-lives) post-production. An aliquot of copper-64 (74 μL, 121 MBq) was buffered by the addition of NaOH solution (0.1 M, 60 μL) followed by NH4OAc buffer (1 M, pH 5.6, 54 μL) to give a final pH of 5-6.

NMR spectra were recorded on an Agilent MR400 NMR (California, USA) (¹H at 400 MHz) at 297 K and referenced in internal solvent residue.

Analytical RP-HPLC traces were recorded using either:
i) an Agilent 1200 HPLC system equipped with an Alltech Hypersil BDS C18 analytical HPLC column (4.6×150 mm, 5 μm) with a flow rate of 1 mL min⁻¹, with UV absorbance being recorded at 214 and 254 nm. Retention times (Rₜ/min) were recorded using a gradient elution of 5-100% B in A (A=0.1% TFA, B=MeCN with 0.1% TFA) over 30 min; or
ii) a Shimadzu LC-20AT system with an SPD-20A UV detector and Lab Logic Flow-RAM Radio HPLC detector. Samples were run on a Phenomenex Luna C18 5 μm 4.6×150 mm column, flow rate 1 mL/min, with a solvent gradient of 5-100% B over 15 min (solvent A: 0.1% TFA in H₂O, solvent B: 0.1% TFA in acetonitrile).

Semi-preparative HPLC was performed on an Agilent 1200 HPLC System using a buffer of A=0.1% TFA and B=0.1% TFA in MeCN with UV detection at 214 nm.

Microwave synthesis was performed using a Biotage (Uppsala, Sweden) Initator+microwave system.

Radio-iTLC analysis was performed on a Lab Logic Scan-RAM PET/SPECT radio-TLC scanner, using silica infused glass microfibre iTLC plates developed using a mobile phase of 10 mM Na₂EDTA in Dulbecco's Phosphate Buffered Saline, origin position at 10 mm, solvent front at 110 mm (120 mm measured in total).

Mass spectrometry was performed on an Orbitrap Exactive Plus with Dionex UltiMate® 3000 (ThermoFisher Scientific).

### Example 1—Mass Spectrometric Analysis of Sar-FAPi

Mass spectrometry was performed on Sar-FAPi. Calculated values: [M+H]+m/z=827.4850, [M+2H]2+m/z=414.2462, [M+3H]3+m/z=276.4999. Detected values: 827.4856, 414.2464, 276.5004.

### Example 2—Radiolabelling

An aliquot of copper-64 (90 μL, approx. 60 MBq) buffered with either PBS or ammonium acetate was added to an aliquot of either Sar-MonoFAPi or Sar-BisFAPi (300 ng, 3 uL from a 10 μg/mL stock in MilliQ H₂O prepared immediately before use). The mixture was allowed to stand at ambient temperature and iTLC performed on a small sample of the reaction mixture. A further 6 μg (0.6 μL from a 0.1 mg/mL stock in MilliQ H₂O prepared immediately before use) was added and TLC after a further 5 mins confirmed quantitative labelling. HPLC analysis was performed immediately afterwards.

### Example 3—Stability Experiments

Samples were analysed by HPLC and iTLC as described in Example 2. The radiolabelled solutions were stored without further dilution at ambient temperature and re-analysed by HPLC and iTLC after 24 hours.

### Example 4—Challenge Experiments

In order to investigate binding of the ligand with a copper ion, the radiolabelled complex was mixed with either cysteine or histidine, which are biologically relevant chelators of copper and will compete with Sar-FAPi for copper ions. Stock solutions of either cysteine hydrochloride and histidine hydrochloride (50 mM in ammonium acetate buffer) were added to [⁶⁴Cu]CuSarFAPi to give a final concentration of 10 mM cysteine or histidine. The reaction mixtures were left at ambient temperature for 1 hour and then analysed by HPLC.

No changes to [⁶⁴Cu]Cu-Sar-FAPi were observed upon addition of either cysteine or histidine, which indicates that the ⁶⁴Cu radioisotope remains chelated in the Sar-FAPi ligand, even in the presence of a competitive chelator.

### Example 5—Cell Binding Studies

SK-MEL-187 human melanoma cells were frozen in aliquots at a density of 30×106 cells per 2 mL screw cap Eppendorf in Media (RPMI+10% foetal calf serum plus 10% DMSO). Cells were thawed at 37 C and resuspended in PBS (2 mL) and transferred to a 10 mL tube. Cells were spun down (2000 rpm for 2 min) and resuspended at 20 million/300 μl. Cells were diluted out to required concentrations, and all samples made up to 300 μl with PBS at the following cell concentrations: 0.5×106, 1×106, 2×106, 5×106, 10×106 and 20×106. Approximately 5 kBq of radio-ligand was added to the medium with activity diluted such that the volume added to assay is 200 μl. Samples were incubated on rotating wheel for 1 hour, then centrifuged (2000 rpm for 2 min) to pellet cells and excess activity (supernatant) removed. Samples were washed with PBS (500 l) and centrifuged again (2000 rpm for 2 min) to pellet cells. Samples were resuspended in PBS (500 l) and cell pellets counted using a gamma counter.

LNCaP C₄₂ human prostate cancer cells were used as a negative control with the same protocol followed. LNCaP C₄₂ cells were frozen in Media (DMEM/F12+10% foetal calf serum plus 10% DMSO).

All methods described herein can be performed in any suitable order unless indicated otherwise herein or clearly contradicted by context. The use of any and all examples, or exemplary language (e.g., “such as”, “i.e.”) provided herein, is intended merely to better illuminate the example embodiments and does not pose a limitation on the scope of the claimed invention unless otherwise claimed. No language in the specification should be construed as indicating any non-claimed element as essential.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

It will be apparent to the person skilled in the art that while the invention is described herein in detail for the purposes of clarity and understanding, various modifications and alterations to the embodiments and methods described herein may be made without departing from the scope of the inventive concept disclosed in this specification.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A compound of Formula (I), or a salt, complex, isomer, solvate or prodrug thereof: 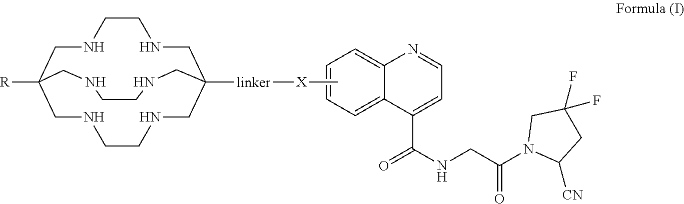
wherein:
R is a group selected from the group consisting of H, OH, halogen, cyano, NO₂, NH₂, optionally substituted C₁-C₁₂ alkyl, optionally substituted amino, optionally substituted amide, optionally substituted aryl and a group having the following structure: 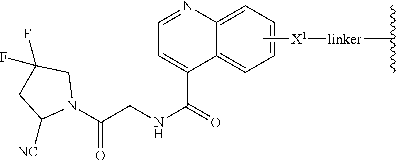
wherein:
X and X¹ may be the same or different and are independently selected from the group consisting of O, S, NH and N—(C₁₋₁₂alkyl).

2. A compound of Formula (I) according to claim 1, wherein the linker comprises one or more moieties selected from the group consisting of:
optionally substituted C₁-C₁₂ alkylene, wherein one or more alkylene groups may be replaced with O, S, NH or N—(C₁₋₁₂alkyl); 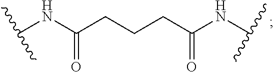 [SMILES: *NC(=O)CCCC(=O)N*]
one or more amino acids; 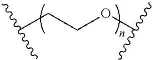
wherein n is an integer from 1 to 10; and

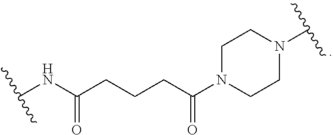 [SMILES: *NC(=O)CCCC(=O)N1CCN(*)CC1]

3. (canceled)

4. A compound of Formula (I) according to claim 1, wherein the linker is selected from the group consisting of: 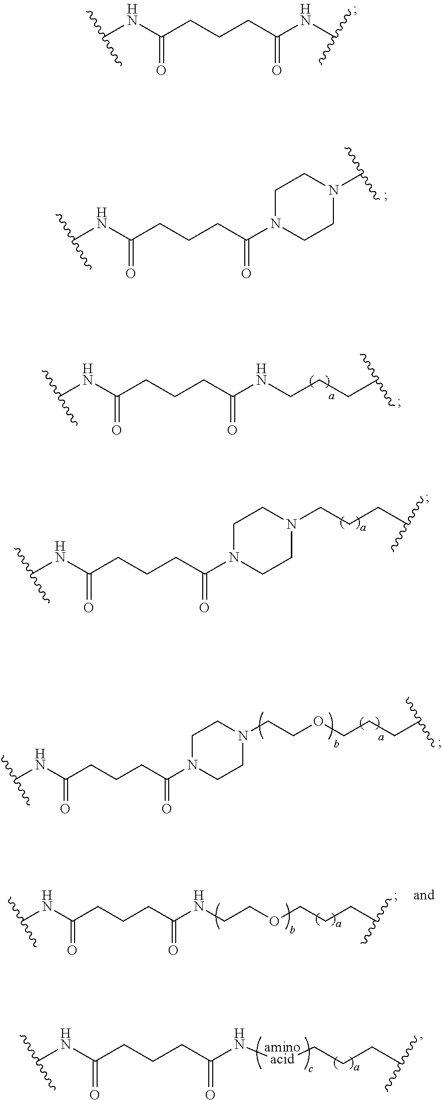
wherein a, b and c are integers independently selected from 1 to 10.

5. A compound of Formula (I) according to claim 1, wherein R is an optionally substituted amide having the following structure: 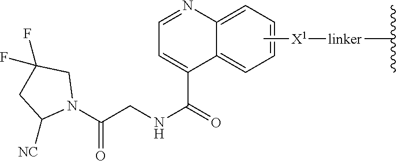
wherein:
X¹ is O, S, NH or N—(C₁₋₁₂alkyl); and
the linker comprises one or more moieties selected from the group consisting of: 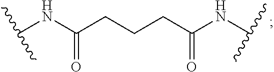 [SMILES: *NC(=O)CCCC(=O)N*]
optionally substituted C₁-C₁₂ alkylene, wherein one or more alkylene groups may be replaced with O, S, NH or N—(C₁₋₁₂alkyl);
one or more amino acids; 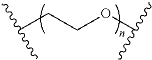
wherein n is an integer from 1 to 10; and

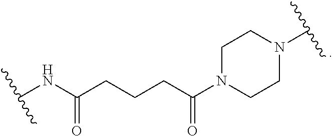 [SMILES: *NC(=O)CCCC(=O)N1CCN(*)CC1]

6. A compound of Formula (I) according to claim 1, wherein the compound of Formula (I) has the structure of Formula (Ia); 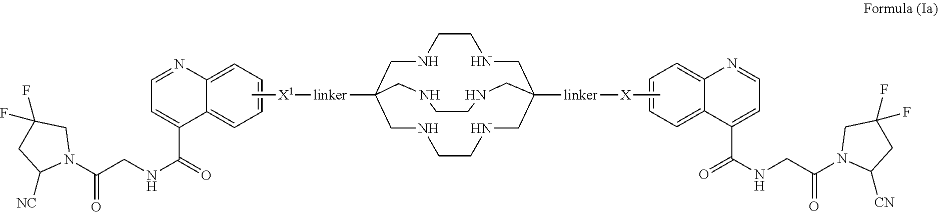
wherein
X and X¹ may be the same or different and are independently selected from the group consisting of O, S, NH and N—(C₁₋₁₂alkyl); and
the linker comprises one or more moieties selected from the group consisting of: 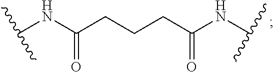 [SMILES: *NC(=O)CCCC(=O)N*]
optionally substituted C₁-C₁₂ alkylene, wherein one or more alkylene groups may be replaced with O, S, NH or N—(C₁₋₁₂alkyl);
one or more amino acids: 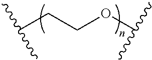
wherein n is an integer from 1 to 10; and

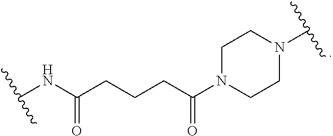 [SMILES: *NC(=O)CCCC(=O)N1CCN(*)CC1]

7. (canceled)

8. A compound of Formula (Ia) according to claim 6, wherein each linker may be the same or different and are independently selected from the group consisting of: 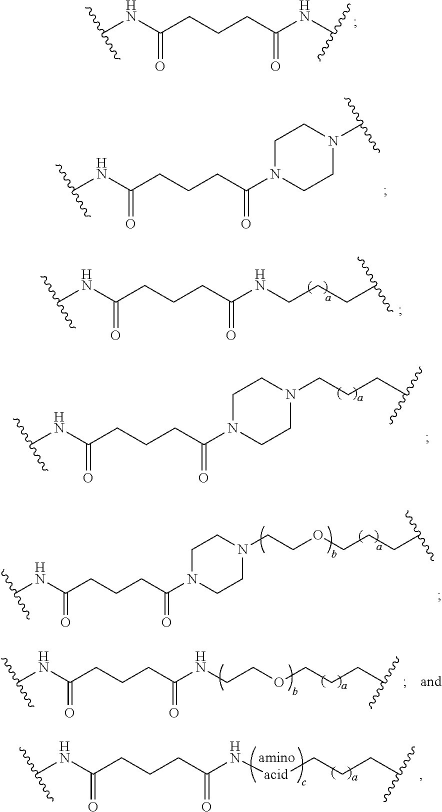
wherein a, b and c are integers independently selected from 1 to 10.

9. A compound of Formula (I) according to claim 1, having one of the following structures.

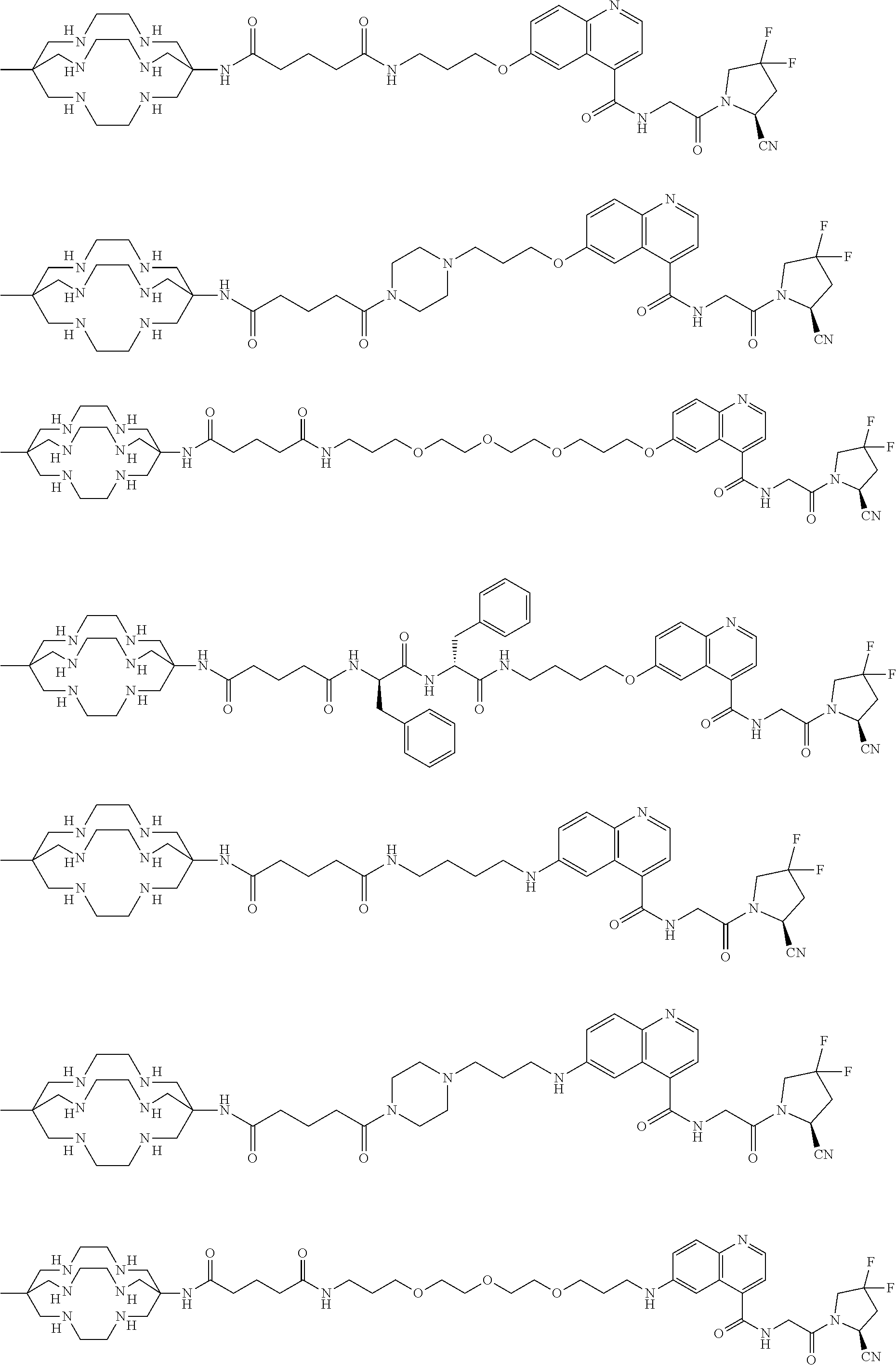

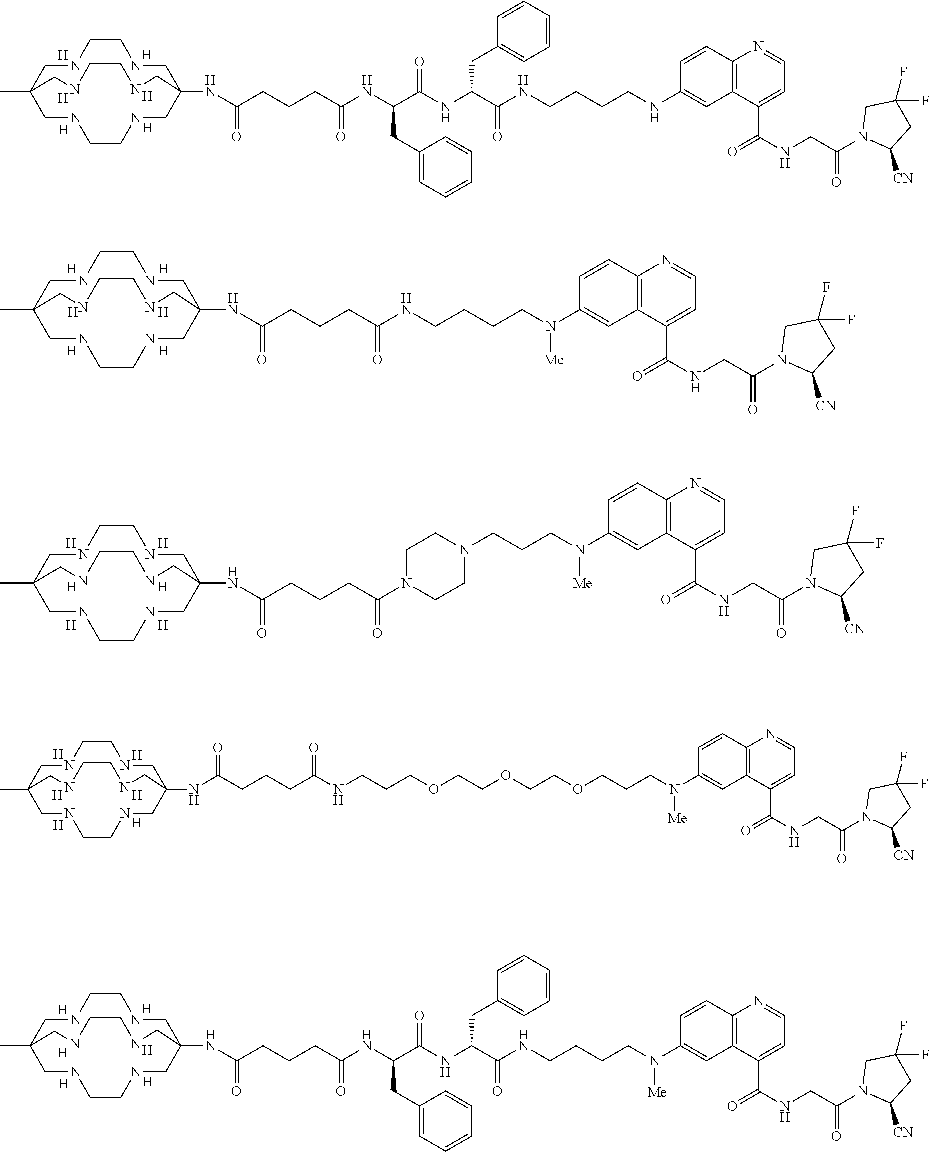

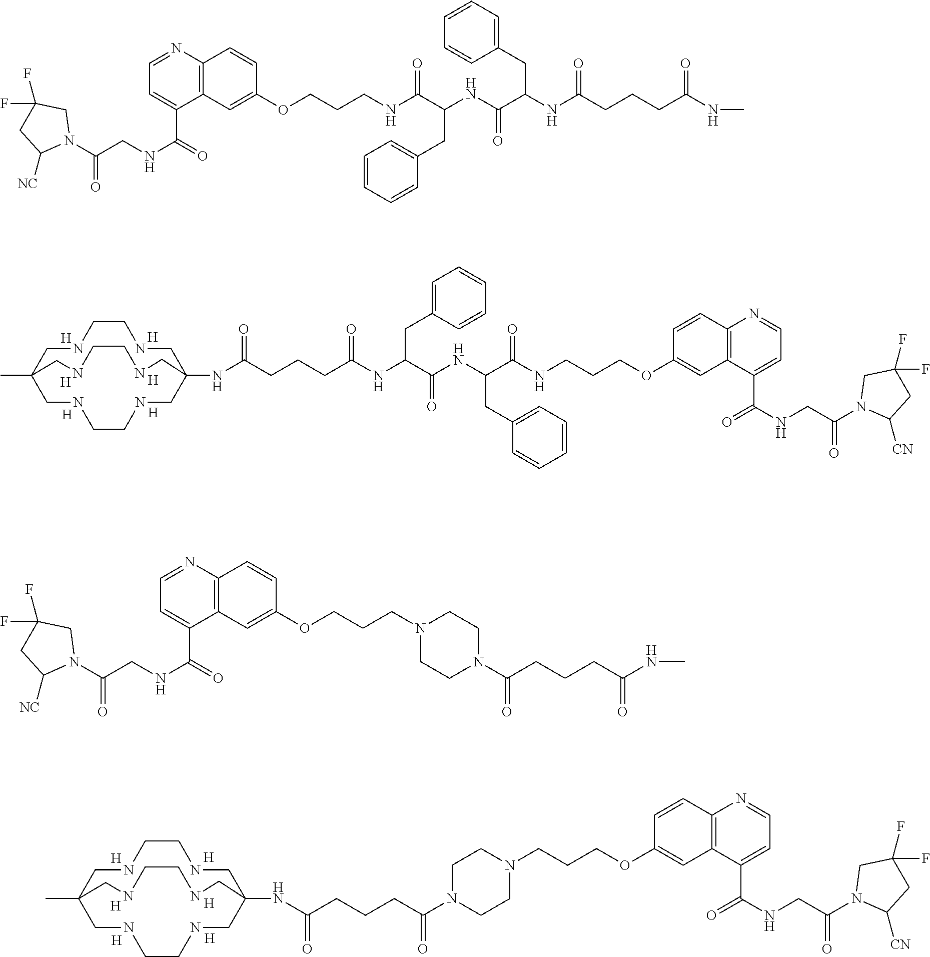

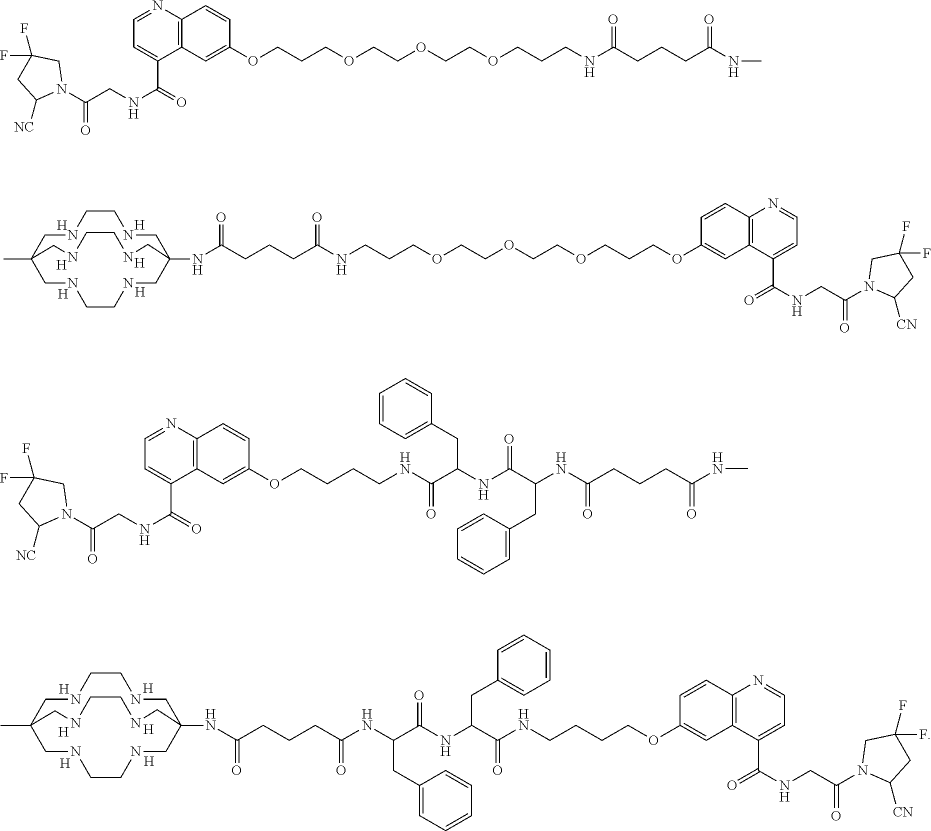

10. A compound according to claim 1, wherein the compound of Formula (I) is Sar-FAPi and has the following structure:

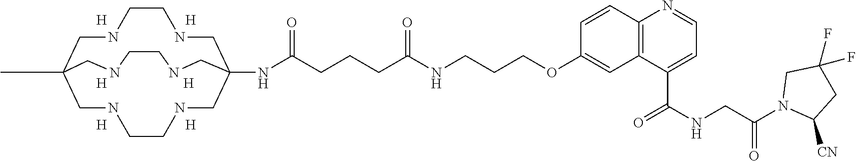

11. A compound according to claim 1, wherein the compound of Formula (Ia) is Sar-bisFAPi and has the following structure:

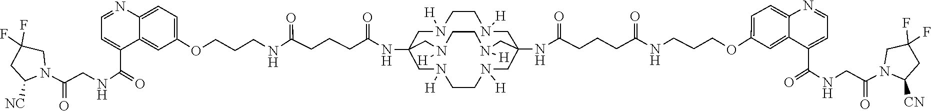 [SMILES: N#C[C@@H]1CC(F)(F)CN1C(=O)CNC(=O)c1ccnc2ccc(OCCCNC(=O)CCCC(=O)NC34CNCCNCC(NC(=O)CCCC(=O)NCCCOc5ccc6nccc(C(=O)NCC(=O)N7CC(F)(F)C[C@H]7C#N)c6c5)(CNCCNC3)CNCCNC4)cc12]

12. A compound of Formula (I) according to claim 1, wherein the compound is complexed with a metal ion, and wherein the metal ion is a Cu radioisotope.

13. (canceled)

14. A compound of Formula (I) according to claim 12, wherein the radioisotope is selected from the group consisting of ⁶⁰ Cu, ⁶¹Cu, ⁶²Cu, ⁶⁴Cu and ⁶⁷Cu.

15. A composition comprising a compound according to claim 1, and a pharmaceutically acceptable excipient.

16. A method for the treatment of a cancer, the method comprising administering to a subject in need thereof, a compound of Formula (I), or a salt, complex, isomer, solvate or prodrug thereof, as defined in claim 1, wherein the compound of Formula (I) contains a suitable radioisotope.

17. A method for radioimaging a cancer, the method comprising administering to a subject in need thereof, the method comprising administering a compound of Formula (I) as defined in claim 1, or a salt, complex, isomer, solvate or prodrug thereof, wherein the compound of Formula (I) contains a suitable radioisotope.

18. A method according to claim 14, wherein the wherein the radioisotope is selected from the group consisting of ⁶⁴Cu, ⁶¹Cu, ⁶²Cu, ⁶⁴Cu and ⁶⁷Cu.

19. A method according to claim 18, wherein the cancer is selected from the group consisting of epithelial ovarian cancer, ovarian carcinoma, osteosarcoma, pancreatic adenocarcinoma, colorectal cancer, lung cancer, non-small cell lung cancer, gastric cancer, endometrial carcinoma, pancreatic adenocarcinoma, medullary thyroid carcinoma, differentiated thyroid cancer, breast cancer, invasive ductal carcinoma of the breast, oral squamous cell carcinoma, esophageal cancer, renal cell cancer, insulinoma, prostate cancer, neuroendocrine differentiated prostate cancer, pheochromocytoma, adenoid cystic cancer, hepatocellular carcinoma, cervical cancer, small intestine cancer, neuroendocrine tumour, anal cancer, chordoma, desmoid tumour, head and neck cancer, thymus cancer, pancreatic cancer, cholangiocellular carcinoma, esophageal cancer, salivary gland cancer, sarcoma, carcinoma of unknown primary cancer.

20. (canceled)

21. A kit comprising:
i) a container comprising a compound of Formula (I) as defined in claim 1, or a pharmaceutically acceptable salt thereof;
ii) a container comprising a solution of a Cu ion; and
instructions for preparing an aqueous formulation of a compound of Formula (I), or a pharmaceutically acceptable salt thereof, complexed with a Cu ion, wherein the Cu ion is a Cu radioisotope.

22. (canceled)

23. A kit according to claim 21, wherein the radioisotope is selected from the group consisting of ⁶⁰ Cu, ⁶¹Cu, ⁶²Cu, ⁶⁴Cu and ⁶⁷Cu.

24. A method for producing a compound of Formula (I) or a pharmaceutically acceptable salt thereof, the method comprising the step of:
i) preparing a compound of Formula A or a protected form thereof: 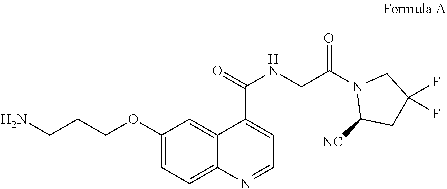 [SMILES: N#C[C@@H]1CC(F)(F)CN1C(=O)CNC(=O)c1ccnc2ccc(OCCCN)cc12]
ii) preparing a compound of Formula B or a protected form thereof: 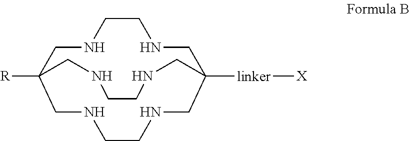
iii) coupling the compound of Formula A or a protected form thereof with the compound of Formula B or a protected form thereof,
to produce the compound of Formula (I) or a protected form thereof,
wherein:
R is a group selected from the group consisting of H, OH, halogen, cyano, NO₂, NH₂, optionally substituted C₁-C₁₂ alkyl, optionally substituted amino, optionally substituted amide, optionally substituted aryl and a group having the following structure: 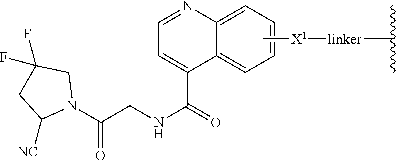
wherein:
X and X¹ may be the same or different and are independently selected from the group consisting of O, S, NH and N—(C₁₋₁₂ alkyl).

25. A method according to claim 15, wherein the wherein the radioisotope is selected from the group consisting of ⁶⁴Cu, ⁶¹Cu, ⁶²Cu, ⁶⁴Cu and ⁶⁷Cu.
